SEQUENCE LISTING (10 sequences)

SEQ ID NO: 1 - protein, 22 aa, Artificial Sequence
  Synthetic
VVAAEEGGRGAAPPKQEFLDIE

SEQ ID NO: 2 - protein, 19 aa, Artificial Sequence
  Synthetic
RSDGSCAWYRGGEEAAVVK

SEQ ID NO: 3 - protein, 20 aa, Artificial Sequence
  Synthetic
VVAAEEGGLWMDWVTEKNIN

SEQ ID NO: 4 - protein, 19 aa, Artificial Sequence
  Synthetic
PLSEWRASYNGGEEAAVVK

SEQ ID NO: 5 - protein, 6 aa, Artificial Sequence
  Synthetic
VVAAEE

SEQ ID NO: 6 - protein, 7 aa, Artificial Sequence
  Synthetic
EEAAVVK

SEQ ID NO: 7 - protein, 14 aa, Artificial Sequence
  Synthetic
RGAAPPKQEFLDIE

SEQ ID NO: 8 - protein, 10 aa, Artificial Sequence
  Synthetic
RSDGSCAWYR

SEQ ID NO: 9 - protein, 12 aa, Artificial Sequence
  Synthetic
LWMDWVTEKNIN

SEQ ID NO: 10 - protein, 10 aa, Artificial Sequence
  Synthetic
PLSEWRASYN

(19) United States
(12) Patent Application Publication
(10) Pub. No.: US 2023/0072949 A1
(43) Pub. Date: Mar. 9, 2023

(54) NANOMATERIALS FOR TARGETED TREATMENT AND IMAGING OF ANEURYSMAL MICROENVIRONMENT

(71) Applicant: The University of North Carolina at Chapel Hill, Chapel Hill, NC (US)

(72) Inventors: Melina R. Kibbe, Chapel Hill, NC (US); Benjamin Ledford, Chapel Hill, NC (US); Nick Tsihlis, Chapel Hill, NC (US)

(21) Appl. No.: 17/759,757

(22) PCT Filed: Jan. 29, 2021
(86) PCT No.: PCT/US2021/015776

§ 371 Date: Jul. 29, 2022

Related U.S. Application Data
(60) Provisional application No. 62/968,896, filed on Jan. 31, 2020.

(51) Int. Cl. A61K 47/64; A61P 9/00

(52) U.S. Cl. CPC A61K 47/645; A61P 9/00

(57) ABSTRACT

Provided herein are compositions and methods for targeted drug delivery to treat aneurysms. In particular, provided herein are nanoscale delivery vehicles for: drugs that inhibit the dilation of an aortic aneurysm, agents that detect abdominal aortic aneurysm by radiographic imaging, and drugs that treat abdominal aortic aneurysm. Also provided here in are methods of generating the nanoscale delivery vehicles and compositions thereof.

### CROSS REFERENCE TO RELATED APPLICATIONS

This application claims priority from and the benefit of U.S. 62/968,896 filed Jan. 31, 2020, which is incorporated herein by reference.

### REFERENCE TO SEQUENCE LISTING

This application includes an electronic sequence listing in a file named 553419SEQLST.txt, created on Jan. 27, 2021 and containing 2,629 bytes, which is hereby incorporated by reference in its entirety for all purposes.

### BACKGROUND

Abdominal aortic aneurysm (AAA) is the most common aortic pathology, and accounts for ˜10,000 deaths annually in the United States. It is a segmental dilation of the abdominal aorta. Treatment typically involves surgical intervention by open surgery or minimally invasive laporascopic repair known as endovascular aneurysm repair (EVAR). However, often when the AAA ruptures, the patient never reaches the hospital or does not survive the surgery. Currently, there are no viable pharmacological options for treating the AAA itself, for example, to reduce AAA enlargement. Further, there is no viable pharmacological treatment for AAA if the aneurysm is large, for example, greater than 5 cm. It is the patient who has AAA that is not large enough to opt for surgical intervention before rupture that lives with the anxiety associated with knowing rupture could occur at any moment.

Presently, pharmacological treatment of AAA and lifestyle management, such as smoking cessation, lowering stress, and the like, has not shown to reduce AAA enlargement. Some therapeutic modalities are only concerned with lowering the risk of a rupture. This involves pharmacological intervention of AAA by managing blood pressure with drugs, such as beta blockers and calcium channel blockers. These drugs can lessen the chance that the aneurysm will rupture or burst. However, in such intervention, the AAA itself is left unresolved and may enlarge, which requires monitoring and concomitant daily anxiety for the patient.

The etiology of AAA is complex. The cause of aneurysmal degeneration is still not fully elucidated. Elevated levels of matrix metalloproteinases (MMP) and fragmented medial elastin have been reported to play a key role in AAA formation.¹⁻⁴ Other mechanisms are known to play a role in AAA formation and rupture. Much more investigation is needed to determine what mechanisms can be targeted with pharmacological approaches.

While many preclinical therapeutics have shown promise, pharmacological interventions have not shown significant inhibition of AAA enlargement.^{5, 6} Further, promising drug candidates for treating AAA may prove to be too toxic or have an unmanageable therapeutic window. Up to now, there has not been a viable means to target AAA pharmacologically. Therefore, a need remains to develop a pharmacological approach to treating and managing AAA.

### BRIEF SUMMARY

Compositions and methods are provided for targeting and treating aneurysms. In one aspect, provided herein are peptide amphiphiles comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; and optionally (e) a therapeutic agent; wherein the hydrophobic non-peptidic segment is covalently attached to the N-terminus (or C-terminus, depending on the molecule orientation) of the β-sheet-forming peptide segment; wherein the β-sheet-forming peptide segment is covalently attached to the targeting moiety; and wherein the charged peptide segment is covalently attached to the targeting moiety. Also provided, are peptide amphiphiles further comprising a therapeutic agent. In embodiments, the aneurysm is an abdominal aortic and/or thoracic aortic and/or peripheral arterial (i.e., iliac, femoral, popliteal, carotid, subclavian, axillary, brachial, renal, splenic, hepatic, etc.) aneurysm.

In another aspect, provided herein are self-assembled nanomaterials comprising a targeting moiety formed of a peptide amphiphile comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; and optionally (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety. Also provided, are self-assembled nanomaterials further comprising a therapeutic agent.

In another aspect, provided herein are methods of inhibiting the dilation of an aortic aneurysm in a subject comprising, administering to the subject a composition comprising a peptide amphiphile comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

In another aspect, provided herein are methods of inhibiting the dilation of an aortic aneurysm in a subject comprising, administering to the subject a composition comprising a self-assembled nanomaterial comprising: a plurality of peptide amphiphiles, wherein said peptide amphiphiles comprise: (a) a hydrophobic non-peptidic segment; (b) a β-sheet-forming peptide segment; (c) a charged peptide segment; (d) a targeting moiety; and optionally (e) a therapeutic agent; wherein the targeting moiety localizes to MMP-2, MT1-MMP, or fragmented elastin; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

In another aspect, provided herein are methods of making peptide amphiphile (PA)-based nanomaterials which target MMP-2, MT1-MMP, or fragmented elastin comprising: synthesizing PA molecules via solid phase peptide synthesis comprising connecting an elastin-targeting peptide, MT1-MMP-targeting peptide, or MMP-2-targeting peptide with a diluent PA backbone; purifying the PA molecules by high-performance liquid chromatography; dissolving targeting PA molecules and the diluent PA in a molar ratio in hexafluoroisopropanol (HFIP); removing the HFIP; and forming the nanomaterials via self-assembly by resuspending the mixture of PA molecules in liquid, such as water or a buffer solution at physiological pH.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates PA Design and TEM. (A) Important peptide amphiphile regions (MMP-2-targeting PA shown). (B) Schematic of typical PA nanomaterial co-assembly. TEM imaging of (C) MMP-2-targeting PA, MT1-MMP-targeting, and elastin-targeting PA nanofibers.

FIG. 2 illustrates AAA Characterization. (A) Photograph of abdominal aorta (indicated with yellow dotted lines) before, and 35 days after CaCl₂ exposure. (B) Mean diameter. (C) Immunofluorescent staining for MMP-2 of suprarenal aorta (uninjured) and infrarenal aorta 35 days following CaCl₂ exposure and mean data. (D) Mean data (MMP-2). (E) Von Kossa stain demonstrating calcification. (F) Verhoeff-Van Gieson stain demonstrating elastin fragmentation. (G) Ultrasound images of infrarenal abdominal aorta. (H) Mean diameter measured via ultrasound.

FIG. 3 illustrates MMP-2 PA targeting/dosage. (A) Gross (top) and histological images of aortas 35 days following AAA induction, 2 hr. after systemic injection. (B) MMP-2-PA dosage study. Red to yellow color (as indicated by the white arrows) indicates PA positive tissue. Red indicates a high amount as seen at the 2.5 mg and 5 mg dose and yellow indicates a lower amount as seen at the 1 mg dose).

FIG. 4 illustrates experimental setup for Example 3. Solid line for dosage and targeting, and dotted lines are localization duration studies.

FIG. 5 illustrates final study design and AAA characterization. (A) AAA study outline of key experimental time points. (B) Photomicrographs of aorta in situ before and 35 days after CaCl₂ exposure. (C) Measurements of aortic diameter before and after CaCl₂) exposure. (D) Immunofluorescence imaging of suprarenal and infrarenal aorta stained for MMP-2. (E) Quantification of MMP-2 staining. (F) Immunofluorescence imaging of suprarenal and infrarenal aorta stained for MT1-MMP. (G) Photomicrographs of suprarenal and infrarenal aorta stained for collagen via Verhoeff-Van Gieson (VVG) stain.

FIG. 6 illustrates peptide amphiphile design and synthesis. (A) Crystal structures used to derive targeting peptides for MMP-2, MT1-MMP, and fragmented elastin. (B) Peptides derived from either contact sites (top 3 panels) or splice variant location (bottom panel). (C) The chemical structure of peptide amphiphile (PA) molecules synthesized containing the derived targeting peptides.

FIG. 7 shows PA material characterization. (A) Conventional transmission electron microscopy (TEM) images of PA co-assemblies consisting of 25 mole % to 95 mole % targeting PA. (C) Small-angle X-ray scattering (SAXS) analysis and (D) wide-angle X-ray scattering analysis of MMP-2 targeting PA co-assemblies.

FIG. 8 illustrates results from the peptide amphiphile targeting study. (A) Light-sheet fluorescence microscopy (LSFM) images of rat aorta 2 hours post PA injection. White color represents tissue autofluorescence, and red to yellow represents the fluorescence of TAMRA-labeled PA. (B) Plotted mean data of the ratio of PA volume to tissue volume for each AAA-targeting PA.

FIG. 9 illustrates targeting PA dosage optimization. (A) LSFM images of rat aorta 2 hours post PA injection of either 2.5 mg, 5 mg, or 10 mg of MMP-2 targeting PA 1. (B) Plotted mean data of the ratio of PA volume to tissue volume for each dose of PA.

FIG. 10 shows MMP-2 targeted PA localization duration. (A) LSFM images of rat aorta 2 hours, 24 hours, 48 hours, and 72 hours post PA injection of 5 mg or 10 mg of MMP-2 targeting PA 1. (B) Plotted mean data of the ratio of PA volume to tissue volume for each dose of PA.

FIG. 11 shows MMP-2 targeting PA predominantly localizes to the aorta. (A) Organ distribution of MMP-2 targeting PA 1 in the kidney, liver, lung, spleen, and aorta of Sprague Dawley rats 2 hours post injection. White color represents autofluorescence and red to yellow represents TAMRA-labeled PA. (B) Plotted mean data of the ratio of PA to tissue in each organ.

FIG. 12 shows differential localization of MMP-2 targeting PA in male and female rats. (A) LSFM images of rat aorta 2 hours post PA injection of 5 mg of MMP-2 targeting PA 1. (B) Plotted mean data of the ratio of PA volume to tissue volume for each sex of animal.

FIG. 13 illustrates CaCl₂ concentration and exposure time optimization. (A) Ultrasound images of abdominal aorta prior to AAA induction and 28 days after induction. (B) Mean data of ultrasound measurements for rats exposed to 0.5 M or 1M CaCl₂ for 20 minutes or 30 minutes. (C) Representative photomicrographs and mean data (D) of rats exposed to various CaCl₂ conditions prior to AAA induction and prior to euthanasia at day 35.

FIG. 14 illustrates complete LSFM of AAA targeting by various PAs. (A) LSFM images of all PA nanofiber formulations tested for AAA targeting capability. White color represents autofluorescence and red to yellow represents TAMRA-labeled PA. (B) Plotted mean data of the ratio of PA nanofiber to tissue volume.

FIG. 15 illustrates exhaustive tissue distribution, representative LSFM images of all rat tissues examined for PA biodistribution.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Overview

Ruptured abdominal aortic aneurysm (AAA) has a very high mortality rate (nearly 90%), and is the most common aortic condition in the United States, taking ˜10,000 lives/year.^{6, 11} Treatment is predominantly limited to relatively high risk surgical intervention.^{6,12} To date, no pharmacological treatments or ongoing clinical trials have provided robust evidence for prevention or reduction of AAA enlargement.^{5, 6} Thus, novel therapeutics are needed.

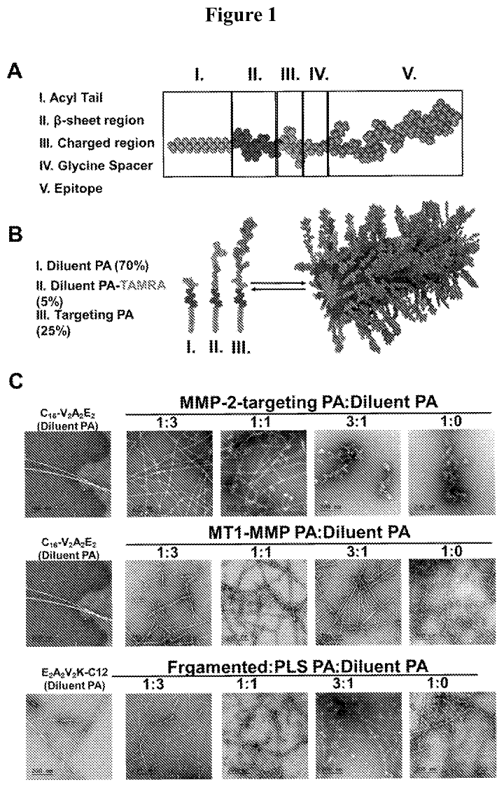

Using targeting moieties, nanoparticles can significantly increase drug efficacy by concentrating drug delivery to diseased tissues. Peptide amphiphile (PA)-based nanomaterials were selected as the targeting and drug delivery vehicle. PA molecules are comprised of an alkyl chain linked to an amino acid sequence which contains a β-sheet forming region, a charged region, and epitope(s) that can target proteins of interest (FIG. 1). This platform was selected because PA molecules spontaneously form high aspect ratio supramolecular nanofibers to increase surface interactions of targeting epitopes while simultaneously serving as a drug delivery vehicle. Also, using intravascularly administered PA nanofibers targeted to prevent restenosis and to stop hemorrhage has been shown to be successful.²²⁻²⁴

Provided herein, a nanoparticle drug delivery system that uses peptide amphiphiles, which spontaneously self-assemble into nanomaterials when placed in an aqueous environment. In embodiments, the aqueous environment is a liquid. In embodiments, the PAs are lyophilized. In embodiments, the PAs are reconstituted in a liquid prior to administration. In embodiments, the PAs are intravenously administered to form nanomaterials that can then target aneurysmal tissue.

Histology of discarded aneurysmal surgical specimens reveals fragmented medial elastin^{2,7,8,13} and increased levels of MMP-2 as a key pathophysiologic finding.^{9, 10, 14-17} Therefore, in embodiments, the PA nanomaterials will target MMP-2, MT1-MMP, or fragmented elastin, which are significantly upregulated in AAA. In embodiments, the PA nanomaterials will target alternative targets such as MT1-MMP, MMP-9, and tenascin-c. In embodiments, the MMP-2-, MT1-MMP-, or fragmented elastin-targeted PAs can also be co-assembled with other non-targeted PAs that are attached to therapeutics via covalent bonds. In embodiments, the attached therapeutic is nitric oxide (NO), angiotensin receptor blockers, ACE inhibitors, MMP inhibitors (e.g., ARP 100, batimastat, etc.), or TGF-β agonists (e.g., N-Acetylpuromycin). In embodiments, the PAs can encapsulate hydrophobic drugs, which normally display poor oral bioavailability, and deliver them directly to the tissue of interest. Also provided herein, a novel biodegradable, biocompatible, intravenous, targeted nanoparticle drug delivery system that localizes to aneurysmal tissue.

In embodiments, the subject matter described herein is directed to an intravenously injectable nanomaterial to target aortic aneurysms. In embodiments, a nanoscale drug delivery system comprised of peptide amphiphile (PA) nanomaterials that can be injected intravenously and target the aneurysmal microenvironment.

In embodiments, the subject matter described herein is directed to nanomaterials comprised of a synthetic peptide covalently attached to an aliphatic molecule. In embodiments, the peptide comprises a β-sheet forming unit, a charged unit, and an optional epitope region. PA molecules spontaneously form nanomaterials in an aqueous environment. Using specific epitopes, the PA molecules can target proteins of interest. The nanofiber structure increases the surface area available to interact with target proteins, which can improve avidity of localization to targeted proteins.

In embodiments, the subject matter described herein is directed to PA molecules that further comprise covalently incorporated therapeutics directly onto PA monomers and/or incorporated hydrophobic drugs into the aliphatic core of the nanofiber structure.

In embodiments, the subject matter described herein is directed to PA nanomaterials that target and deliver therapeutics. Multiple PA monomers can be co-assembled with both targeting PAs and PA monomers containing covalently attached therapeutics, fluorescent tags, and/or contrast agents.

In embodiments, the subject matter described herein is directed to PA molecules incorporating epitopes to target specific proteins overexpressed in aneurysms (matrix metalloproteinase 2 [MMP-2] and membrane type metalloproteinase 1 [MT1-MMP]), and fragmented elastin, which is a feature exclusive to pathological arteries.

In embodiments, the subject matter described herein is directed to PA nanomaterials comprising covalently incorporated nitric oxide-releasing molecules directly into the PA monomers, which were co-assembled with targeting monomers. Upon co-assembly these supramolecular nanomaterials target diseased tissue and activate localized release of nitric oxide.

In embodiments, the subject matter described herein is directed to PA nanomaterials comprising microbubbles, covalently incorporated positron-emitting nuclides such as fluorine-18 (¹⁸F), and/or gadolinium contrast agents into the PA structure. Such PA nanomaterials can be useful as contrast agents to visualize early stage aneurysms before significant dilation occurs.

### II. Definitions

As used herein, the term “nanofiber” refers to an elongated or threadlike filament (e.g., having a significantly greater length dimension than width or diameter) with a diameter of less than 100 nanometers.

As used herein, the term “nanosphere” refers to an approximately spherical globular shape having approximately (e.g., a <25% difference, <10% difference, <5% difference) the same diameters in the x, y, and z dimensions, with a diameter of less than 500 nanometers (e.g., <200 nm, <100 nm, etc.).

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the term “nanomaterial” refers to nanofibers, nanospheres, micelles, nanoribbons, and a variety of other structures that can be formed as a result of supramolecular interactions. In certain embodiments, the supramolecular interactions are between the peptide amphiphiles and other components in the nanomaterial.

As used herein, the term “physiological conditions” refers to the range of conditions of temperature, pH, and tonicity (or osmolality) normally encountered within tissues in the body of a living human.

As used herein, the terms “self-assemble,” “self-assembled,” and “self-assembly” refer to formation or product of a discrete, non-random, aggregate structure from component parts; said assembly occurring spontaneously through random movements of the components (e.g., molecules) due only to the inherent chemical or structural properties and attractive forces of those components. A “self-assembled nanofiber” refers to a product comprised of a plurality of peptide amphiphiles. As used herein, a “plurality” refers to two or more peptide amphiphiles.

As used herein, the term “peptide amphiphile” refers to a molecule that, at a minimum, includes a non-peptide lipophilic (hydrophobic) segment, a structural peptide segment, and optionally a functional peptide segment. The peptide amphiphile may express a net charge at physiological pH, either a net positive or negative net charge, or may be zwitterionic (i.e., carrying both positive and negative charges). Certain peptide amphiphiles consist of or comprise four segments: (1) a hydrophobic, non-peptidic segment comprising an acyl group of six or more carbons, (2) a β-sheet-forming peptide segment; (3) a charged peptide segment, and (4) a targeting moiety (e.g., targeting peptide).

As used herein and in the appended claims, the term “lipophilic component” or “hydrophobic component” refers to the acyl moiety disposed on the N-terminus (or C-terminus, depending on the orientation) of the peptide amphiphile. This lipophilic segment may be herein and elsewhere referred to as the aliphatic, lipophilic, or hydrophobic segment. The hydrophobic component should be of a sufficient length to provide amphiphilic behavior and micelle (or nanosphere or nanofiber) formation in water or another polar solvent system.

Accordingly, in the context of the embodiments described herein, the hydrophobic component preferably comprises a single, linear acyl chain of the formula: Cₙ₋₁H₂ₙ₋₁C(O)— where n=6-22. A particularly preferred single, linear acyl chain is the lipophilic group, palmitic acid. However, other small lipophilic groups may be used in place of the acyl chain.

As used herein, the term “structural peptide” or “β-sheet-forming peptide” refers to the intermediate amino acid sequence of the peptide amphiphile molecule between the hydrophobic segment and the charged peptide segment of the peptide amphiphile. This “structural peptide” or “β-sheet-forming peptide” is generally composed of three to ten amino acid residues with non-polar, uncharged side chains, selected for their propensity to form a β-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form β-sheets). However, non-naturally occurring amino acids of similar β-sheet forming propensity may also be used. Peptide segments capable of interacting to form β-sheets and/or with a propensity to form β-sheets are understood (see, e.g., Mayo et al. Protein Science (1996), 5:1301-1315; herein incorporated by reference in its entirety). In a preferred embodiment, the N-terminus of the structural peptide segment is covalently attached to the oxygen of the lipophilic segment and the C-terminus of the structural peptide segment is covalently attached to the N-terminus of the charged peptide segment.

As used herein, the term “charged peptide segment” refers to the intermediately disposed peptide sequence between the structural peptide segment or β-sheet forming segment and the functional peptide. In some embodiments, the charged segment provides for solubility of the peptide amphiphile in an aqueous environment, and preferably at a delivery location within a cell, tissue, organ, or subject. The charged peptide segment contains two or more amino acid residues that have side chains that are ionized under physiological conditions, examples of which selected from the 20 naturally occurring amino acids include Lys (K), Arg (R), Glu (E), and/or Asp (D), along with other uncharged amino acid residues. Non-natural amino acid residues with ionizable side chains could be used, as will be evident to one ordinarily skilled in the art. This segment may be from about 2 to about 7 amino acids long, and may be comprised of about 3 or 4 different amino acids. The charged peptide segment may include those amino acids and combinations thereof which provide this solubility and permit self-assembly and is not limited to polar amino acids such as E or K and combinations of these for modifying the solubility of the peptide amphiphile.

One or more Gly (G) residues may be added to the “charged peptide segment,” intermediately disposed between the charged residues and the functional peptide segment (e.g., targeting peptide). While not wishing to be bound by theory, the inclusion of one or more Gly (G) residues appears to prevent salt-bridge formation between the Glu and the Lys amino acid side-chains by altering side-chain orientation of these residues relative to each other, improving solubility of the peptide in salt solutions of similar composition to extracellular fluid. In one embodiment, the charged peptide segments have the formula (E)ₓ(G)_{y}, wherein x is 2 to 6 and y is 1 to 6. In another embodiment, the charged peptide segment has 2 to 4 Glu (E) residues and 1 to 2 Gly (G) residues. In another aspect, the charged peptide segment has 2 Glu (E) residues and 1 Gly (G) residue. In yet another aspect of the invention, the charged peptide segment has 3 Glu (E) residues and 1 Gly (G) residue. In another embodiment, the charged peptide segment has 4 Glu (E) residues and 1 Gly (G) residue. The glycine residues may also act as a spacer to provide greater accessibility of the targeting peptide to the protein of interest by extending the targeting peptide past the surface of the nanomaterial.

As used herein, the term “targeting peptide” refers to amino acid sequences which mediate the localization (or retention) of sequences, molecules, or supramolecular complexes associated therewith to a particular location or locations (e.g., sub-cellular location (e.g., organelle), an organ (e.g., heart), tissue (e.g., cardiovascular tissue), or localized with a receptor or binding partner for the targeting peptide)). Peptide amphiphiles and structures (e.g., nanofibers) bearing targeting peptides have been reported to congregate in desired locations based on the identity and presence of the targeting peptide. A targeting peptide described in exemplary embodiments herein is the MMP-2- or MT1-MMP or fragmented elastin-targeting peptide. Such targeting peptides have been shown to deliver targeted nanomaterials comprising such peptides to aneurysmal tissue. In embodiments, the targeting peptide targets MMP-2- or MT1-MMP or fragmented elastin by binding to and/or localizing to MMP-2- or MT1-MMP or fragmented elastin. In embodiments, the targeted nanomaterial binds to and/or localizes to sites where MMP-2- or MT1-MMP or fragmented elastin is expressed. In embodiments, the sites are aneurysmal sites.

Proteins are said to have an “N-terminus” and a “C-terminus.” The term “N-terminus” relates to the start of a protein or polypeptide, terminated by an amino acid with a free amine group (—NH₂). The term “C-terminus” relates to the end of an amino acid chain (protein or polypeptide), terminated by a free carboxyl group (—COOH).

“Sequence identity” or “identity” in the context of two polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid, or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gin | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | s | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., amino acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “fragment” when referring to a protein means a protein that is shorter or has fewer amino acids than the full-length protein. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### III. Compositions of Matter

A. Peptide Amphiphile (PA)-Based Nanomaterials

Peptide amphiphiles (Hartgerink et al. P Natl Acad Sci USA 99, 5133 (2002); Hartgerink et al. Science 294, 1684 (2001); herein incorporated by reference in their entireties) (PAs) are a class of self-assembling molecules that are composed of a hydrophobic segment conjugated to a sequence of amino acids. PAs can form long, high aspect ratio cylindrical filaments in water and have been studied for a range of applications in regenerative medicine (Mata et al., Biomaterials 31, 6004 (2010); Shah et al., P Natl Acad Sci USA 107, 3293 (2010); Huang et al. Biomaterials 31, 9202 (2010); Webber et al., P Natl Acad Sci USA 108, 13438 (2011); herein incorporated by reference in their entireties). PA bioactivity is derived from presentation of peptide sequences on the surface of self-assembled nanostructures that form in solution. The rheological properties of these materials can be tuned by concentration and peptide sequence (Pashuck et al. Journal of the American Chemical Society 132, 6041 (2010); herein incorporated by reference in its entirety).

Examples of peptide amphiphile (PA)-based nanomaterials discussed herein can be found in U.S. Pat. No. 9,517,275; herein incorporated by reference in its entirety. In some embodiments, provided herein are peptide amphiphiles comprising: (a) a hydrophobic non-peptidic segment; (b) a β-sheet-forming peptide segment: (c) a charged peptide segment; (d) a targeting moiety, and (e) a therapeutic agent. In some embodiments, the hydrophobic non-peptidic segment is covalently attached to the N-terminus of the β-sheet-forming peptide segment; wherein the C-terminus of the β-sheet-forming peptide segment is covalently attached to the N-terminus of the charged peptide segment; and wherein the C-terminus of the charged peptide segment is covalently attached to the N-terminus of the targeting moiety. In some embodiments, the hydrophobic non-peptidic segment is covalently attached to the C-terminus of the β-sheet-forming peptide segment: wherein the N-terminus of the β-sheet-forming peptide segment is covalently attached to the C-terminus of the charged peptide segment; and wherein the N-terminus of the charged peptide segment is covalently attached to the C-terminus of the targeting moiety. In some embodiments, the hydrophobic non-peptidic segment comprises an acyl chain. In some embodiments, the acyl chain comprises C₆-C₂₄ (e g., C₆ . . . C₈ . . . C₁₀ . . . C₁₂ . . . C₁₄ . . . C₁₆ . . . C₁₈ . . . C₂₀ . . . C₂₂ . . . C₂₄) In some embodiments, the acyl chain comprises lauric acid. In some embodiments, the β-sheet-forming peptide segment comprises AAVV. In some embodiments, the charged peptide segment comprises a plurality of Lys (K), Arg (R), Glu (E), and/or Asp (D) residues. In some embodiments, the charged peptide segment comprises 2-7 amino acids in length with 50% or more amino acids selected from Lys (K), Arg (R), Glu (F), and/or Asp (D) residues. In some embodiments, the charged peptide segment comprises KK. In some embodiments, the targeting moiety comprises a targeting sequence for a protein of interest. In some embodiments, the target protein is MMP-2, MT1-MMP, or fragmented elastin. In some embodiments, the therapeutic agent is covalently linked to the peptide amphiphile. In some embodiments, the therapeutic agent is nitric oxide (NO). In some embodiments, the NO is covalently linked to the peptide amphiphile as a nitroso group. In some embodiments, the nitroso group is attached via nitrosylation of a cysteine residue. In some embodiments, the peptide amphiphile contains an S-nitrosylated cysteine residue.

In some embodiments, provided herein are self-assembled nanomaterials formed of the peptide amphiphiles described above (or elsewhere herein). In some embodiments, the nanofiber has a diameter of less than 200 nm (e g., <150 nm, <100 nm, <50 nm) In some embodiments, the nanofiber has a diameter of 10-200 nm (e.g., 20-180 nm, 50-200 nm, 30-150 nm, or other ranges less than 200 nm and greater than 10 nm). In some embodiments, the nanofiber has a length of at least 1 μm. In some embodiments, the nanofiber has a length of at least 500 nm to 50 μm (e.g., >500 nm, >1 μm, >2 μm, >5 μm, >10 μm, <50 μm, <40 μm, <30 μm, <20 μm, etc.).

In some embodiments, provided herein are supramolecular nanostructures (e.g., formed by self-assembly of a single molecule type) that target the site of vascular injury and deliver therapeutic (e.g., NO). An exemplary molecular building block for the supramolecular nanostructures is a peptide amphiphile (PA) containing a peptide segment conjugated to an aliphatic tail. This broad family of molecules is in the creation, assembly, and/or manufacture of bioactive nanostructures for regenerative medicine and drug delivery (Cui, et al. Biopolymers, Vol. 94 1-18 (2010); Matson & Stupp. Chem. Commun, Vol. 48 26 (2011); Webber, M. J., et al. Proceedings of the National Academy of Sciences, Vol. 108 13438-13443 (2011); Matson, et al. Soft Matter, Vol. 8 6689 (2012); Soukasene, S., et al. ACS Nano, Vol. 5 9113-9121 (2011); herein incorporated by reference in their entireties). PAs are made to self-assemble into nanostructures of various shapes, including spheres and fibers, by altering the peptide sequences (Muraoka et al. Angew. Chem. Int. Ed., Vol. 48 5946-5949 (2009); Cui et al. Nano Lett., Vol. 9 945-951 (2009); Paramonov et al. J. Am. Chem. Soc., Vol. 128 7291-7298 (2006); herein incorporated by reference in their entireties). This ability is attractive to vascular applications because a filamentous shape has been previously shown to extend circulation time and bind to the endothelium (Geng, Y., et al. Nature Nanotechnology, Vol. 2 249-255 (2007); Shuvaev, V. V., et al. ACS Nano, Vol. 5 6991-6999 (2011); herein incorporated by reference in their entireties). The peptide portion of a PA is also an ideal site to integrate various bioactive functions.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus of the peptide, in order to create the lipophilic segment. Synthesis typically starts from the C-terminus, to which amino acids are sequentially added using either a Rink amide resin (resulting in an —NH₂ group at the C-terminus of the peptide after cleavage from the resin), or a Wang resin (resulting in an OH group at the C-terminus). Accordingly, embodiments described herein encompasses peptide amphiphiles having a C-terminal moiety that may be selected from the group consisting of —H, —OH, —COOH, —CONH₂, and —NH₂.

The lipophilic segment is typically incorporated at the N-terminus of the peptide after the last amino acid coupling and is composed of a fatty acid or other acid that is linked to the N-terminal amino acid through an acyl bond. Additionally, the lipophilic segment can be incorporated at the C-terminus via an acyl bond to a lysine side chain. In aqueous solutions, PA molecules self-assemble (e.g., into cylindrical micelles (a.k.a., nanofibers)) that bury the lipophilic segment in their core and display the functional peptide on the surface. The structural peptide undergoes intermolecular hydrogen bonding to form β-sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl tail) segment of sufficient length (e.g., >3 carbons, >5 carbons, >7 carbons, >9 carbons, etc.) is covalently coupled to peptide segment (e.g., an ionic peptide having a preference for β-strand conformations) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture. In various embodiments, hydrophobic segments pack in the center of the assembly with the peptide segments exposed to an aqueous or hydrophilic environment to form cylindrical nanostructures that resemble filaments. Such nanofilaments display the peptide regions on their exterior and have a hydrophobic core.

To induce self-assembly of an aqueous solution of peptide amphiphiles, the pH of the solution may be changed (raised or lowered) or multivalent ions, such as calcium, or charged polymers or other macromolecules may be added to the solution. Though not intending to be bound by theory, self-assembly is facilitated in the instant case by the neutralization or screening (reduction) of electrostatic repulsion between ionized side chains on the charged peptide segment.

In some embodiments, the hydrophobic segment is a non-peptide segment (e.g., alkyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, aromatic segments, pi-conjugated segments, etc.

In some embodiments, peptide amphiphiles comprise a targeting moiety. In particular embodiments, a targeting moiety is the C-terminal most segment of the PA. In some embodiments, the targeting moiety is attached to the C-terminal end of the charged segment. In some embodiments, the targeting moiety is exposed on the surface of an assembled PA structure (e.g., nanofiber). A targeting moiety is typically a peptide (e.g., targeting peptide), but is not limited thereto. For example, in some embodiments, a targeting moiety is a small molecule (e.g., the target for a receptor, a ligand for a protein, etc.). Examples described in detail herein utilize a peptide sequence that localizes to MMP-2, MT1-MMP, or fragmented elastin as a targeting moiety. The presence of the MMP-2, MT1-MMP, or fragmented elastin targeting sequence directs the PA structures (e.g., nanofibers) to the aneurysmal tissue, allowing them to localize at the site of interventions (e.g., to isolate the therapeutic action at the desired site). Further, targeting moieties may localize to (and thereby direct PA structures to) proteins or other targets that are localized in other regions of the body, or even subcellular locations. Targeting moieties may direct PA structures (and therefore the therapeutics attached thereto or encapsulated therein) to specific organs, tissues, cell types, subcellular locations (e.g., organelles), pathogens (e.g., viruses, bacteria, etc.), diseases (e.g., to cancerous cells), etc. Targeting peptides and other moieties for achieving such localization are understood. As additional targeting moieties are discovered, they too may find use in embodiments described herein.

Suitable peptide amphiphiles, PA segments, PA nanostructures, and associated reagents and methods are described, for example in U.S. Pat. Nos. 8,512,693; 8,450,271; 8,138,140; 8,124,583; 8,114,835; 8,114,834; 8,080,262; 8,063,014; 7,851,445; 7,838,491; 7,745,708; 7,683,025; 7,554,021; 7,544,661; 7,534,761; 7,491,690; 7,452,679; 7,390,526; 7,371,719; 6,890,654; herein incorporated by reference in their entireties.

In certain embodiments, peptide amphiphiles further comprise a therapeutic group. In some embodiments, a therapeutic (e.g., a drug that prevents proliferation and neointimal hyperplasia (e.g., NO)) is covalently or non-covalently attached to PA. For example, a therapeutic is attached to a PA such that it is exposed on the surface of the assembled PA structure (e.g., nanofiber). In some embodiments, a therapeutic is covalently linked to the peptide portion of the PA. In some embodiments, any suitable chemistry known to those in the art is used for the covalent attachment (e.g., modification of a cysteine in the PA (e.g., S-nitrosylation)). In embodiments, the therapeutic agent is covalently attached via a hydrophobic interaction. In other embodiments, a therapeutic is attached to PA such that it is released (e.g., in a burst, over time, upon exposure to particular conditions, etc.) from the PA and/or assembled PA structure (e.g., nanofiber). In some embodiments, a therapeutic is not attached to the individual PAs, but is incorporated into or encapsulated within a PA supramolecular structure. In such embodiments, the therapeutic is released from the structure at a desired rate and/or under desired conditions (e.g., physiological conditions, upon localization of the targeting moiety to a target, etc.).

Exemplary therapeutic groups include small molecules (e.g., NO), peptides, antibodies, nucleic acids (e.g., siRNA, antisense RNA, etc.), etc. Examples described in detail herein utilize nitric oxide as a therapeutic. In the examples, PAs were S-nitrosylated (e.g., SNO groups added to the PAs). Upon degradation of the SNO groups, NO is released from the assembled PA structure (e.g., nanofiber). Therapeutic delivery of NO is not limited to S-nitrosylation of PAs. Further, embodiments are not limited to delivery of NO. Any therapeutic that can be delivered and localized to a desired site of action (e.g., by a targeting moiety) finds use in embodiments described herein. For example, drugs that prevent proliferation and neointimal hyperplasia may be delivered to sites of arterial intervention to reduce and/or prevent restenosis in the cardiovascular system. Exemplary drugs for such use include, but are not limited to: nitric oxide, acetylsalicylic acid, rapamycin, paclitaxel, etc.

The characteristics (e.g., shape, rigidity, hydrophilicity, etc.) of a PA supramolecular structure depend upon the identity of the components of a peptide amphiphile (e.g., lipophilic segment, charged segment, structural segment, functional segment, etc.). For example, nanofibers, nanospheres, intermediate shapes, and other supramolecular structures are achieved by adjusting the identity of the PA component parts. In examples provided herein, the fiber shape of the nanoscale delivery vehicle proved particularly conducive to cardiovascular applications, and exhibited significant and measurable advantage over, for example nanosphere delivery vehicles. In other embodiments, for example, when a different site of action is desired, other vehicle characteristics may be desirable. In some embodiments, provided herein are nanoscale delivery vehicles with tunable shapes to best suit the intended therapeutic delivery location. For example, nanofibers may be preferred over nanospheres for a particular delivery site (e.g., site of vascular intervention). Likewise, in some embodiments, a particular length to diameter ratio (or range of ratios) is particularly advantageous for a delivery location.

In certain embodiments, PAs and the nanofibers assembled therefrom comprise a targeting moiety configured to deliver the PA and/or nanomaterial to a desired location within a cell, tissue, organ, body system, or subject (e.g., human, non-human primate, rodent, etc.). In some embodiments, a PA and/or nanomaterial is also associated with (e.g., covalently or non-covalently) a therapeutic agent configured for action at the site to which the PA and/or nanomaterial is localized. In exemplary embodiments described herein an MMP-2-, MT1-MMP-, or fragmented elastin-targeting sequence that is part of a PA is used to localize a nanomaterial covalently linked to nitric oxide to a site of intervention of a subject. Embodiments are not limited to such conditions (e.g., AAA, dilation of aortic aneurysms), targeting moieties (e.g., aneurysmal tissue targeting; MMP-2, MT1-MMP, or fragmented elastin targeting; etc.), or therapeutics (e.g., NO). One of skill in the art will understand how to select and test combinations of therapeutic agents and targeting moieties for prevention and/or treatment of a variety of diseases and conditions. For example, a PA comprising tumor targeting peptides and linked to chemotherapeutics finds use in the treatment of cancer. Likewise, PAs comprising peptides targeting clotting factors and linked to antithrombic agents find use in the treatment or prevention of stroke and/or other cardiovascular conditions. Embodiments find use, for example, in the treatment or prevention of any disease or condition where systemic administration of a therapeutic, followed by localization to a treatment site, is desired.

B. Peptide Amphiphile (PA)-Based Nanomaterials that Target MMP-2, MT1-MMP, or Fragmented Elastin

In embodiments, a nanotechnology targeted to aneurysmal tissue, to eventually serve as a therapeutic delivery platform to prevent/regress AAA formation.

Three target proteins, MMP-2, MT1-MMP, and fragmented elastin, each being key features of AAA were selected. MMP-2 is critical for aneurysm development in rodent knockout models.⁴ MMP-2 makes an attractive target for AAA tissue because it is produced by smooth muscle cells of the media, and it is expressed at high levels throughout aneurysm development.^{3,9,10,17,26} Using contact sites between MMP-2 or MT1-MMP and tissue inhibitor of metalloproteinase 2 (TIMP-2), we designed peptides to target MMP-2 or MT1-MMP (FIG. 1C). Our third target takes advantage of fragmentation of elastin fibers which expose the amorphous elastin core for targeting during AAA progression. Prior efforts to target elastin have relied on antibodies.²⁷ We target fragmented elastin with peptides found in the elastin-binding domain of the 67-kDa elastin binding protein (EBP).²⁸

Here, multiple epitopes containing supramolecular nanomaterials self-assembled from peptide amphiphile (PA) molecules are disclosed. The embodiments take advantage of three key features of the pathophysiology of aneurysm formation: upregulation of MMP-2/MT1-MMP and fragmentation of elastin.⁷⁻¹⁰ PA nanomaterials are designed to specifically target MMP-2/MT1-MMP and fragmented elastin in aneurysmal tissue, and this nanotechnology is biocompatible. Examples of peptides designed to target MMP-2/MT1-MMP and fragmented elastin are in Table 1 and Table 2.

In one embodiment, a peptide capable of targeting an epitope of MMP-2 that has at least 95% identity with a sequence selected from SEQ ID NOs: 1-3. In another embodiment, a peptide capable of targeting an epitope of MMP-2 that has at least 80%, at least 85%, at least 87%, at least 89%, at least 90%, at least 91%, at least 92%, at least 93%, at least 94%, at least 95%, at least 96%, at least 97%, at least 98%, at least 99%, or 100% identity with a sequence selected from SEQ ID NOs: 1-4. In embodiments, a peptide that is capable of targeting an epitope on MT1-MMP. In one embodiment, a peptide that is capable of targeting an epitope on fragmented elastin that has at least 95% identity with a SEQ ID NO: 4. In another embodiment, a peptide that is capable of targeting an epitope on fragmented elastin that has at least 80%, at least 85%, at least 87%, at least 89%, at least 90%, at least 91%, at least 92%, at least 93%, at least 94%, at least 95%, at least 96%, at least 97%, at least 98%, at least 99%, or 100% identity with SEQ ID NO: 4.

TABLE 1
Peptides designed to target MMP-2/MT1-MMP and fragmented elastin.

| | | | Efficiency of | |
|---|---|---|---|---|
| Target | PA ID | PA Sequence | Synthesis | Targeting Capability |
| MMP-2 | MMP-2_RGA | (C₁₆)VVAAEEGGRG | High yield | Targets aneurysm |
| | | AAPPKQEFLDIE | High purity | |
| | | (SEQ ID NO: 1) | | |
| | MMP-2_RSD | RSDGSCAWYRGGE | Low yield | Not tested due to low |
| | | EAAWK(C₁₂) (SEQ | Moderate | synthesis yield |
| | | ID NO: 2) | purity | |
| MT1-MMP | MTI-MMP_LWM | (C₁₆)VVAAEEGGLW | High yield | Targets aneurysm |
| | | MDWVTEKNIN | High purity | |
| | | (SEQ ID NO: 3) | | |
| Fragmented | Fragmented | PLSEWRASYNGGE | High yield | Does not target |
| Elastin | Elastin_PLS | EAAVVK(C₁₂) (SEQ | High purity | aneurysm |
| | | ID NO: 4) | | |

TABLE 2
PA sequence component parts.

| | | | Targeting |
|---|---|---|---|
| Sequence | Backbone | Linker | Peptide |
| SEQ ID NO: 1 | (C₁₆)VVAAEE | GG | RGAAPPKQEFLDIE |
| | (SEQ ID NO: 5) | | (SEQ ID NO: 7) |
| SEQ ID NO: 2 | EEAAVVK(C12) | GG | RSDGSCAWYR |
| | (SEQ ID NO: 6) | | (SEQ ID NO: 8) |
| SEQ ID NO: 3 | (C16)VVAAEE | GG | LWMDWVTEKNIN |
| | (SEQ ID NO: 5) | | (SEQ ID NO: 9) |
| SEQ ID NO: 4 | EEAAVVK(C₁₂) | GG | PLSEWRASYN |
| | (SEQ ID NO: 6) | | (SEQ ID |
| | | | NO: 10) |

### IV. Therapeutic Methods

The peptide amphiphile (PA)-based nanomaterials disclosed herein can be used in various methods. For example, they can be used in methods of treating abdominal aortic aneurysms (AAA) in a subject, in methods of inhibiting the dilation of aortic aneurysms in a subject, and in methods of detecting high levels of MT1-MMP and MMP-2 early before dilation has occurred and then treating the same.

A method of inhibiting the dilation of aortic aneurysms in a subject can comprise, for example, administering to the subject a composition comprising a peptide amphiphile comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; and optionally (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety. The therapeutic agent can be, for example, nitric oxide (NO).

A method of treating an aortic aneurysm, including an abdominal aortic aneurysm in a subject can comprise, for example, administering to the subject a composition comprising a peptide amphiphile comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; and optionally (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety. The therapeutic agent can be, for example, nitric oxide (NO).

A method of treating an aortic aneurysm, including an abdominal aortic aneurysm in a subject can comprise, administering to the subject a composition comprising a self-assembled nanomaterial comprising: a plurality of peptide amphiphiles, wherein said peptide amphiphiles comprise: (a) a hydrophobic non-peptidic segment; (b) a β-sheet-forming peptide segment; (c) a charged peptide segment; (d) a targeting moiety; and optionally (e) a therapeutic agent; wherein the targeting moiety localizes to MMP-2, MT1-MMP, or fragmented elastin; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

The term “inhibiting the dilation” refers to (i) treats or prevents the dilation, (ii) attenuates, ameliorates, or eliminates the worsening, such as growth, of the dilation, or (iii) prevents or delays the onset of dilation. In certain embodiments, the inhibiting is relative to the expected result if no peptide amphiphile or self-assembled nanomaterial is administered.

The term “treat” or “treating” refers to both therapeutic treatment and prophylactic or preventative measures, wherein the object is to prevent or lessen the aortic aneurysm. Treating may include one or more of directly affecting or curing, suppressing, inhibiting, preventing, reducing the severity of, delaying the onset of, slowing the progression of, stabilizing the progression of, reducing/ameliorating symptoms associated with the aortic aneurysm, or a combination thereof.

The term “subject” refers to a mammal (e.g., a human) in need of therapy for, or susceptible to developing, an aortic aneurysm. The term subject also refers to a mammal (e.g., a human) that receives either prophylactic or therapeutic treatment. The subject may include dogs, cats, pigs, cows, sheep, goats, horses, rats, mice, non-human mammals, and humans. The term “subject” does not necessarily exclude an individual that is healthy in all respects and does not have or show signs of an aortic aneurysm.

Pharmaceutical formulations comprising peptide amphiphiles or peptide amphiphile (PA)-based nanomaterials can be prepared for parenteral administration, e.g., bolus or intravenous injection and the like with a pharmaceutically acceptable parenteral vehicle and in a unit dosage injectable form. Peptide amphiphiles or peptide amphiphile (PA)-based nanomaterials are optionally mixed with one or more pharmaceutically acceptable excipients (Remington's Pharmaceutical Sciences (1980) 16^{th} edition, Osol, A. Ed.). Peptide amphiphiles or peptide amphiphile (PA)-based nanomaterials (and any additional therapeutic agent) can be administered by any suitable means, including parenteral, intravenous, intraarterial, intrapulmonary, and the like.

### V. Methods of Making

Methods of making the peptide amphiphile (PA)-based nanomaterials disclosed herein. A method of making peptide amphiphile (PA)-based nanomaterials which target MMP-2, MT1-MMP, or fragmented elastin can comprise, for example, synthesizing PA molecules via solid phase peptide synthesis comprising connecting an elastin-targeting peptide or MMP-2-targeting peptide with a diluent PA backbone; purifying the PA molecules by high-performance liquid chromatography; dissolving targeting PA molecules and the diluent PA in a molar ratio in hexafluoroisopropanol (HFIP); removing the HFIP; and forming the nanomaterials via self-assembly by resuspending the mixture of PA molecules in liquid, such as water or a buffer solution at physiological pH. In embodiments, the liquid is a biological liquid such as blood. In embodiments, the PA molecules are lyophilized. In embodiments, the PA molecules are reconstituted in a liquid prior to administering to a subject.

In embodiments, an elastin-targeting peptide is covalently incorporated into a diluent PA backbone, for example, E₂A₂V₂K(C₁₂) (SEQ ID NO: 6). In embodiments, an MMP-2-targeting peptide is covalently incorporated into a diluent PA backbone, for example, C₁₆-V₂A₂E₂ (SEQ ID NO: 5).

In embodiments, each targeting PA is further co-assembled with a diluent PA. In embodiments, the optimal parameters for nanomaterial assembly (molar ratios that allow for the best fiber formation, modification of buffer solutions, heating and cooling (e.g., annealing) the PA solutions, or allowing the solutions to sit at room temperature or 4° C. (e.g., aging), etc.), as well as the critical aggregation concentration are determined.

The molar ratio of the targeting PA to the diluent PA (e.g., C₁₆-V₂A₂E₂ or E₂A₂V₂K(C₁₂)) can be from about 1:100 to about 100:1; or from about 1:50 to about 50:1; or from about 1:10 to about 10:1; about 1:9 to about 9:1; or from about 1:5 to about 5:1; or from about 1:2 to about 2:1; or about 1:1. In embodiments, the molar ratio is about 1:9 to about 9:1. In embodiments, the diluent PA backbone and diluent PA are the same. In embodiments, the diluent PA backbone and diluent PA are different.

Specific embodiments described herein include:

1. A peptide amphiphile comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; and optionally (e) a therapeutic agent; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

2. The peptide amphiphile of embodiment 1, wherein said targeting moiety comprises a peptide capable of targeting an epitope of MMP-2, an epitope of MT1-MMP, or an epitope of fragmented elastin.

3. The peptide amphiphile of embodiment 2, wherein said targeting moiety is a peptide that is capable of targeting an epitope on MMP-2.

4. The peptide amphiphile of embodiment 3, wherein said peptide comprises a sequence with at least 80% homology SEQ ID NO: 1.

5. The peptide amphiphile of embodiment 2, wherein said targeting moiety is a peptide that is capable of targeting an epitope on MT1-MMP.

6. The peptide amphiphile of embodiment 5, wherein said peptide comprises a sequence with at least 80% homology to SEQ ID NO: 3.

7. The peptide amphiphile of embodiment 2, wherein said targeting moiety comprises a peptide that is capable of targeting an epitope on fragmented elastin.

8. The peptide amphiphile of any one of embodiments 1-7, further comprising a therapeutic agent.

9. The peptide amphiphile of embodiment 8, wherein the therapeutic agent is nitric oxide.

10. The peptide amphiphile of embodiment 8, wherein the therapeutic agent is an angiotensin receptor blocker.

11. The peptide amphiphile of embodiment 8, wherein the therapeutic agent is an ACE inhibitor.

12. The peptide amphiphile of embodiment 8, wherein the therapeutic agent is an MMP inhibitor.

13. The peptide amphiphile of embodiment 8, wherein the therapeutic agent is a TGF-β agonist.

14. The peptide amphiphile of embodiment 1, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

15. A self-assembled nanomaterial comprising:
a plurality of peptide amphiphiles, wherein said peptide amphiphiles comprise:
   (a) a hydrophobic non-peptidic segment;
   (b) a β-sheet-forming peptide segment;
   (c) a charged peptide segment; and
   (d) a targeting moiety, wherein the targeting moiety localizes to MMP-2, MT1-MMP, or fragmented elastin;
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

16. The self-assembled nanomaterial of embodiment 15 wherein said targeting moiety comprises a peptide capable of targeting an epitope of MMP-2, an epitope of MT1-MMP, or an epitope of fragmented elastin.

17. The self-assembled nanomaterial of embodiment 16, wherein said targeting moiety is a peptide that is capable of targeting an epitope on MMP-2.

18. The self-assembled nanomaterial of embodiment 17, wherein said peptide comprises a sequence with at least 80% homology SEQ ID NO: 1.

19. The self-assembled nanomaterial of embodiment 16, wherein said targeting moiety is a peptide that is capable of targeting an epitope on MT1-MMP.

20. The self-assembled nanomaterial of embodiment 19, wherein said peptide comprises a sequence with at least 80% homology to SEQ ID NO: 3.

21. The peptide amphiphile of embodiment 16, wherein said targeting moiety comprises a peptide that is capable of targeting an epitope on fragmented elastin.

22. The self-assembled nanomaterial of embodiment 15-21, further comprising a therapeutic agent.

23. The self-assembled nanomaterial of embodiment 22, wherein the therapeutic agent is nitric oxide.

24. The self-assembled nanomaterial of embodiment 22, wherein the therapeutic agent is an angiotensin receptor blocker.

25. The self-assembled nanomaterial of embodiment 22, wherein the therapeutic agent is an ACE inhibitor.

26. The self-assembled nanomaterial of embodiment 22, wherein the therapeutic agent is an MMP inhibitor.

27. The self-assembled nanomaterial of embodiment 22, wherein the therapeutic agent is a TGF-β agonist.

28. The self-assembled nanomaterial of embodiment 15, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

29. A method of inhibiting the dilation of an aneurysm in a subject comprising, administering to the subject a composition comprising a self-assembled nanomaterial comprising:
a plurality of peptide amphiphiles, wherein said peptide amphiphiles comprise:
   (a) a hydrophobic non-peptidic segment;
   (b) a β-sheet-forming peptide segment;
   (c) a charged peptide segment; and
   (d) a targeting moiety,
wherein the targeting moiety targets MMP-2 or MT1-MMP or fragmented elastin; wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

30. The method of embodiment 29, wherein the aneurysm is an aortic aneurysm, a thoracic aortic, or a peripheral arterial aneurysm.

31. The method of embodiment 30, wherein the aortic aneurysm is an abdominal aortic aneurysm.

32. The method of embodiment 30, wherein the peripheral arterial aneurysm is an iliac aneurysm, a femoral aneurysm, a popliteal aneurysm, a carotid aneurysm, a subclavian aneurysm, an axillary aneurysm, a brachial aneurysm, a renal aneurysm, a splenic aneurysm, or a hepatic aneurysm.

33. The method of embodiment 29, wherein the peptide amphiphile further comprises a therapeutic agent.

34. A method of delivering a therapeutic agent to an aneurysm site in a subject comprising, administering to the subject a composition comprising a self-assembled nanomaterial comprising:
a plurality of peptide amphiphiles, wherein said peptide amphiphiles comprise:
   (a) a hydrophobic non-peptidic segment;
   (b) a β-sheet-forming peptide segment;
   (c) a charged peptide segment; and
   (d) a targeting moiety, wherein the targeting moiety localizes to MMP-2 or MT1-MMP or fragmented elastin;
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

35. A method of making a peptide amphiphile (PA)-based nanomaterial which targets MMP-2, MT1-MMP, or fragmented elastin comprising:
synthesizing targeting PA molecules via solid phase peptide synthesis comprising contacting an elastin-targeting peptide, MMP-2-targeting peptide, or MT1-MMP-targeting peptide with a diluent PA backbone;
purifying the PA molecules;
dissolving targeting PA molecules and with a diluent PA in a molar ratio in a solvent; removing the solvent; and
forming the nanomaterial via self-assembly by resuspending the mixture of PA molecules in liquid at physiological pH.

36. The method of embodiment 35, wherein the solvent is hexafluoroisopropanol (HFIP).

37. The method of embodiment 35, wherein the liquid is water or a buffer solution.

38. The method of embodiment 35, wherein the nanomaterial is a nanofiber.

39. The method of embodiment 35, wherein the PA molecules are purified by high-performance liquid chromatography.

40. The method of embodiment 35, wherein the molar ratio is about 1:9 to about 9:1.

41. The method of embodiment 35, wherein the diluent PA backbone and the diluent PA are the same.

42. The method of embodiment 35, wherein the diluent PA backbone and the diluent PA are different.

43. The method of embodiment 35, wherein the elastin-targeting peptide, MMP-2-targeting peptide, or MT1-MMP-targeting peptide is connected to the diluent PA backbone by a covalent bond in the resulting targeting PA molecule.

The disclosed subject matter is further described in the following non-limiting Examples. It should be understood that these Examples, while indicating preferred embodiments of the invention, are given by way of illustration only.

### EXAMPLES

### Example 1: Studies on Elastin-Targeting PA and MMP-2-Targeting PA

After identifying the peptide sequences described above, we covalently incorporated a putative elastin-targeting peptide into a diluent PA (E₂A₂V₂K(C₁₂)) and an MMP-2-targeting peptide into another diluent PA backbone (C₁₆-V₂A₂E₂) through solid phase peptide synthesis. We then co-assembled the PA molecules in molar ratios with their respective diluent PA to aid in nanofiber formation, and fluorescently labeled diluent PA to aid in visualization of PAs in cell culture and in vivo studies (FIG. 1B). Transmission electron microscopy (uranyl acetate staining) revealed nanofiber formation with both PA molecules (FIG. 1C). The elastin-targeting PA formed fibers at all co-assembly ratios, and the MMP-2-targeting PA formed fibers up to a 1:1 ratio with the diluent PA.

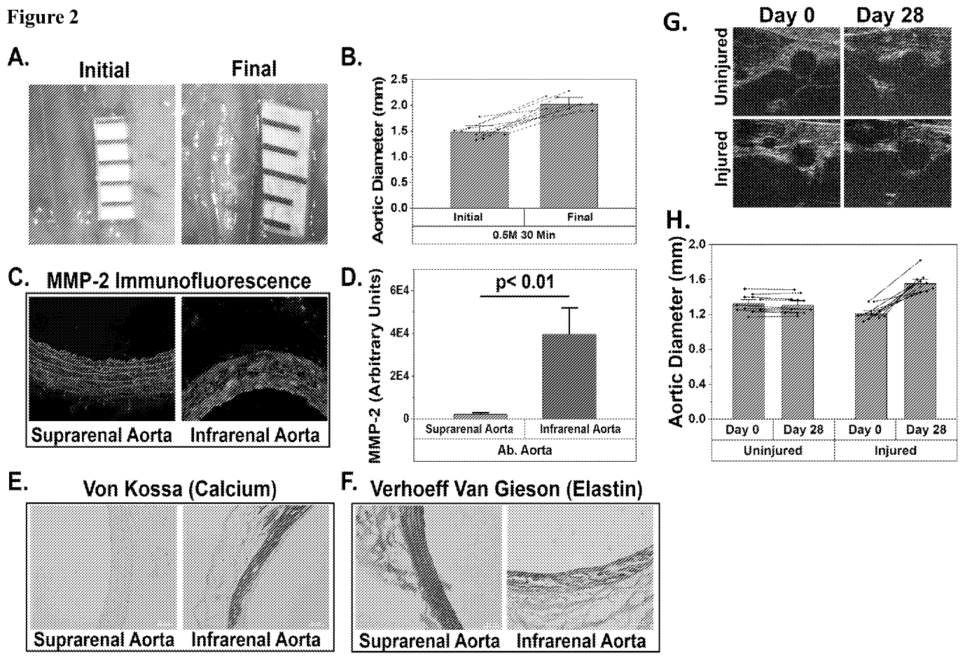

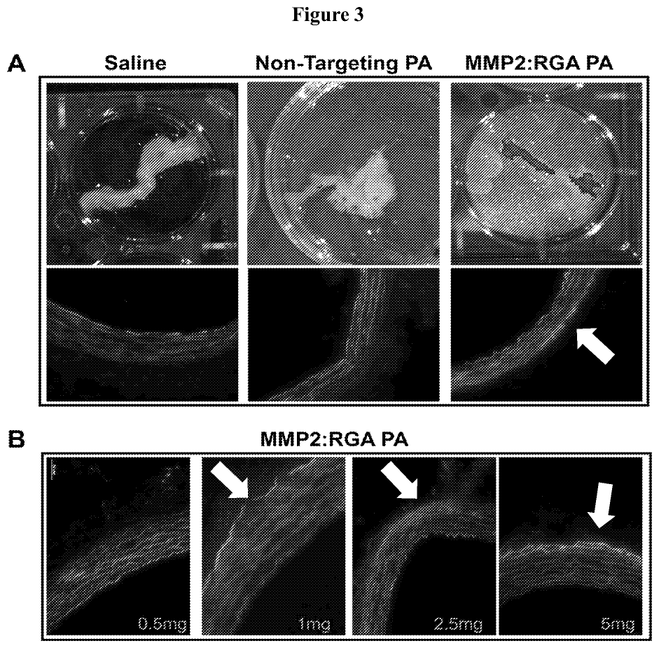

We also validated the CaCl₂ model, by inducing aneurysms in 10 male and 7 female Sprague Dawley rats. We measured aortic diameter before AAA induction and then at days 14, 21, and 28 after induction using ultrasound. At day 35, we performed an endpoint surgery and measured the diameter of the aorta, perfusion-fixed the rats, and collected tissue. Tissue was cryo-sectioned and assayed for elastin (Verhoeff-Van Gieson (VVG)), calcium (Von Kossa), and MMP-2 (IF) staining. Measurements of the aorta revealed an average dilation of >35% by day 28 (data not shown) and >40% by day 35 (FIG. 2B). There was no significant difference between male and female rats in aortic dilation (data not shown). Further, we found both elastin fragmentation, and robust MMP-2 protein staining in the aortas exposed to CaCl₂, with negligible amounts of fragmentation and MMP-2 in the suprarenal aortas from the same animals (FIG. 2 C+F). Fragmentation of elastin and MMP-2 corresponded to regions of the aorta that were calcified (FIG. 2E). Lastly, we produced preliminary targeting data for our MMP-2-targeting PA, showing localization to areas of the aorta that have high levels of MMP-2 (FIG. 3).

### Example 2: Design, Synthesize, and Characterize the Structural Properties and Biocompatibility of PA Nanomaterials Targeted to MMP-2 and Elastin In Vitro

After identifying peptide sequences that target MMP-2, MT1-MMP, or fragmented elastin, PAs were synthesized via solid phase peptide synthesis and purified by high-performance liquid chromatography. Nanofiber formation was characterized by circular dichroism spectroscopy, small-angle X-ray scattering, and transmission electron microscopy. Each targeting PA was co-assembled with diluent PA to determine the optimal parameters for nanomaterial assembly, as well as the critical aggregation concentration. The biocompatibility of the PA nanomaterials in vitro was characterized by assessing endothelial and vascular smooth muscle viability, proliferation, and targeting ability using immunofluorescence (IF) to co-localize the targeted PA with MMP-2, MT1-MMP, and elastin.

Experimental Design: Design and synthesize PA nanomaterials targeted to MIP-2, MT1-MMP, and fragmented elastin. Peptide sequences were selected to target proteins unique to aneurysmal pathology. For MMP-2, 2 peptides were designed based on regions of surface contact from X-ray crystallographic structures of TIMP-2 in complex with MMP-2. For MT1-MMP, 1 peptide was designed based on a region of surface contact from X-ray crystallographic structures of TIMP-2 in complex with MT1-MMP. For fragmented elastin, modeling software was used to determine sites containing secondary structure in the tropoelastin binding domain of EBP and 3 targeting peptides are designed.²⁸⁻³² These peptides were incorporated into PAs via solid phase peptide synthesis.

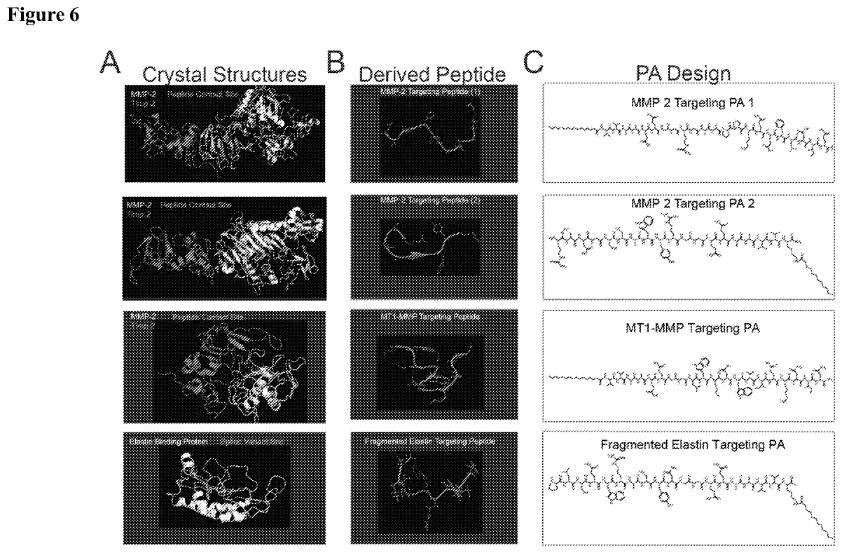

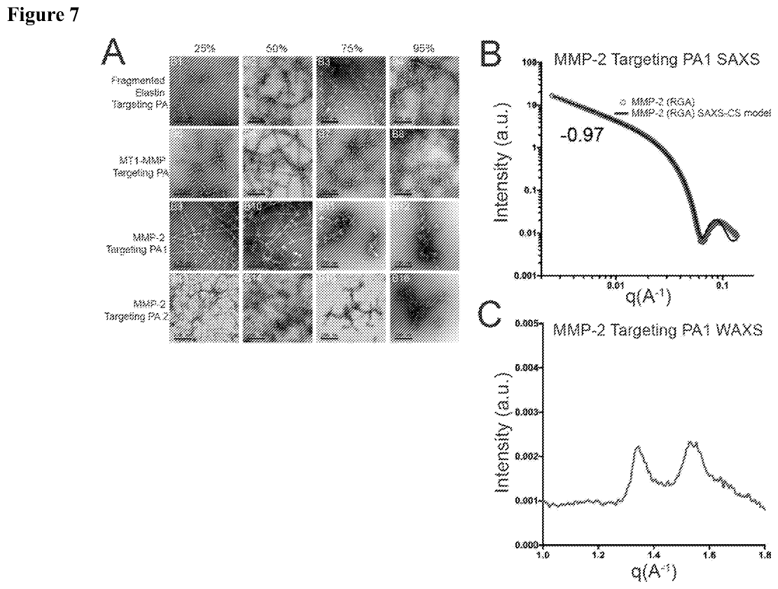

Characterization of the physical properties of targeting PA molecules. We identified peptide sequences from proteins that are known to target to our proteins of interest,^{30,33} and we covalently incorporated the peptides into PA molecules that form nanofibers (FIG. 1). The nanomaterial formation was optimized and higher co-assembly ratios of both targeting PAs were stabilized. Several methods to improve nanofiber formation are reported in the literature. The effects of annealing at elevated temperatures during nanofiber formation and aging the PAs were examined.³⁴ After nanofiber formation was optimized, secondary structure was further characterized with circular dichroism (CD) spectroscopy, PA dimensions with small-angle X-ray scattering (SAXS), wide-angle X-ray scattering (WAXS), and critical aggregation concentration. PA molecules synthesized are shown in FIG. 6. Characterization of MMP-2-targeting PA 1 is shown in FIG. 7.

PA cytocompatibility. The biocompatibility of PA nanomaterials in vitro is characterized by exposing endothelial and vascular smooth muscle to PA nanomaterials and measure cell viability (calcein ethidium staining), proliferation (EdU staining), cellular uptake (confocal microscopy), and localization of the fluorescently labeled PAs (confocal microscopy). As an analog for fragmented elastin, we will use dermal fibroblasts obtained from fibrillin-1 knockout mice to recapitulate the fragmented disruption of fibrillin-1 observed in elastin fragmentation.³⁵ In embodiments, toxicity associated with either PA can be addressed by using an alternate diluent PA for that nanomaterial, as there is great flexibility in the sequences for the charged and β-sheet regions used to form nanomaterials.³⁴ High levels of PA nanofibers localized to cell membranes has been reported to increase cytotoxicity particularly with positively charged PAs.³⁶ Although this is unlikely since we are using diluent PAs with negative charge, we could see excessive localization of either of our PA nanofibers to the cell surface. In embodiments, the amount of charge present on the diluent PA is decreased. In another embodiment, an entirely different backbone PA is used. In embodiments, PAs that target MMP-2, MT1-MMP, or fragmented elastin are designed and synthesized using different targeting peptide sequences, as different sequences can impact the biocompatibility. In embodiments, alternative targets are used for designing targeting peptides including MMP-9 and tenascin-c.

### Example 3: Evaluate the Targeting Specificity of the Targeted PA Nanomaterials to Aneurysmal Tissue In Vivo

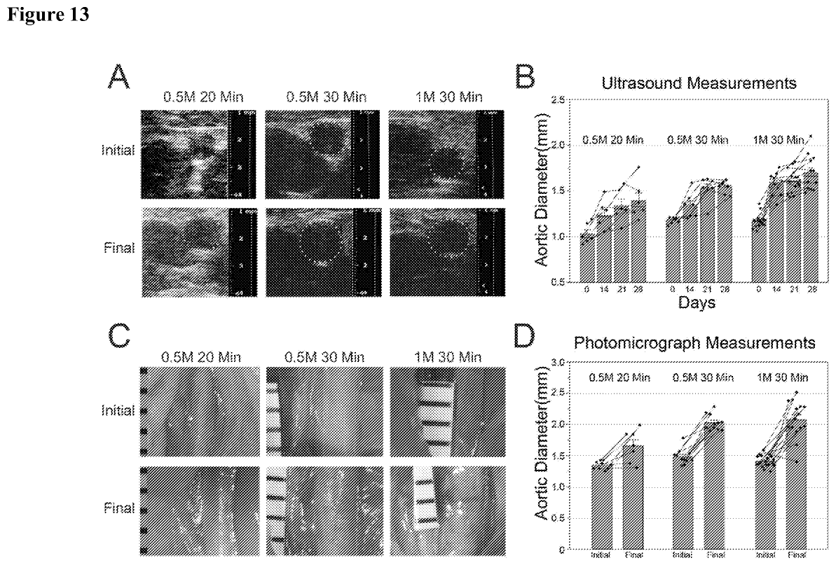

To evaluate the targeting specificity of the targeted PA nanomaterials, we induced AAA in the infrarenal aorta of Sprague Dawley rats by exposure to CaCl₂. CaCl₂) concentration and exposure time optimization is shown in FIG. 13. After 35 days, a period consistent with aneurysm formation, the targeted PA nanomaterials was injected intravenously. We assessed targeting specificity and duration to aneurysmal sites as well as to all vital organs. We also determined optimal concentration, dose, and co-assembly ratios. Controls included rats injected with saline alone, non-targeted PA nanomaterials, and PA nanomaterials bearing a scrambled targeting sequence.

The clinical translation potential of the PA nanomaterials is tested by targeting capability in an in vivo model of AAA formation. The most specific and biocompatible MMP-2- and elastin-targeted nanomaterials from above are tested.

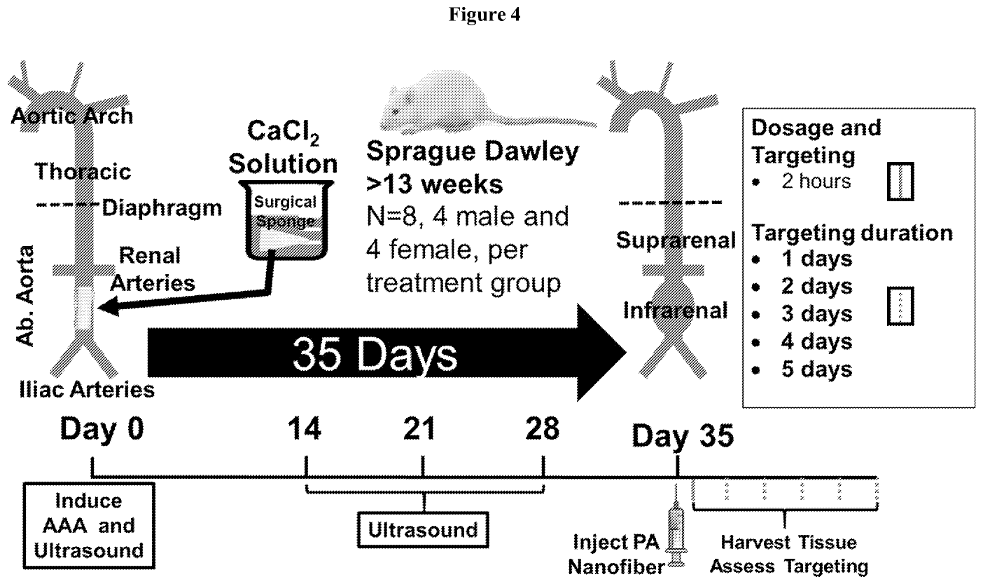

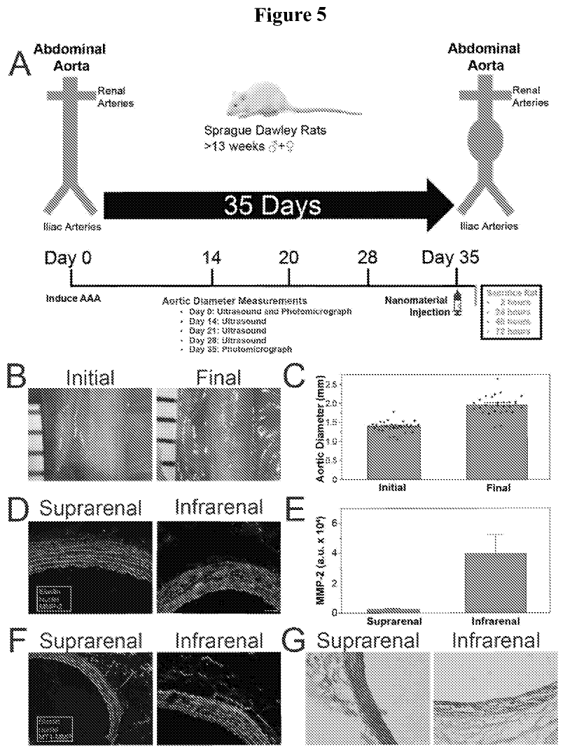

Experimental Design. Dosage and targeting specificity. To evaluate specific localization of targeted PA nanomaterials, AAA is induced in the infrarenal aorta of male and female Sprague Dawley rats by exposure to CaCl₂) (FIG. 4 and FIG. 5A). The features of human AAA can be recapitulated in rodent models. Here, we developed and validated a surgical model of AAA, where CaCl₂ is applied to the periadventitial surface of the aorta.^{4, 18} While no single model perfectly replicates human AAA, similarities between CaCl₂-induced AAA and human aneurysms makes this a useful model. For instance, both human and CaCl₂) induced AAA led to fragmentation of medial elastin and increased MMP-2 levels.^{6, 19-21}

Aortic dilation is measured using ultrasound imaging. At day 35 after CaCl₂) exposure, a time point we have established produced significant aortic dilation (FIGS. 2A and 2B), increased levels of MMP-2 (FIG. 2C), and elastin fragmentation (FIG. 2E), rats received one of 12 treatments via intravenous tail vein injection (n=8 rats, 4 males and 4 females, per treatment group). For the elastin-targeting PA: 1) 5 mg of 25 mole %, 2) 5 mg of 50 mole %, and 3) 5 mg of 95 mole %, for the MT1-MMP-targeting PA: 4) 5 mg of 25 mole %, 5) 5 mg of 50 mole %, and 6) 5 mg of 95 mole %, for the MMP-2-targeting PA 1 25 mole % doses of 7) 2.5 mg, 8) 5 mg, and 9) 10 mg, and the following controls: 10) 5 mg of altered hydrogen bonding MMP-2-control PA 25 mole %, 11) 5 mg of scrambled MMP-2-control PA 25 mole %, and 12) saline. Rats are euthanized 2 hours post PA injection using inhaled isoflurane anesthesia followed by thoracotomy.

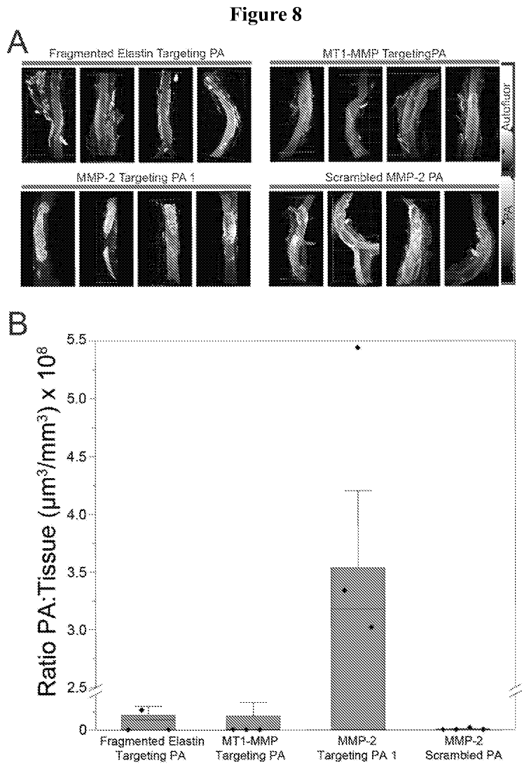

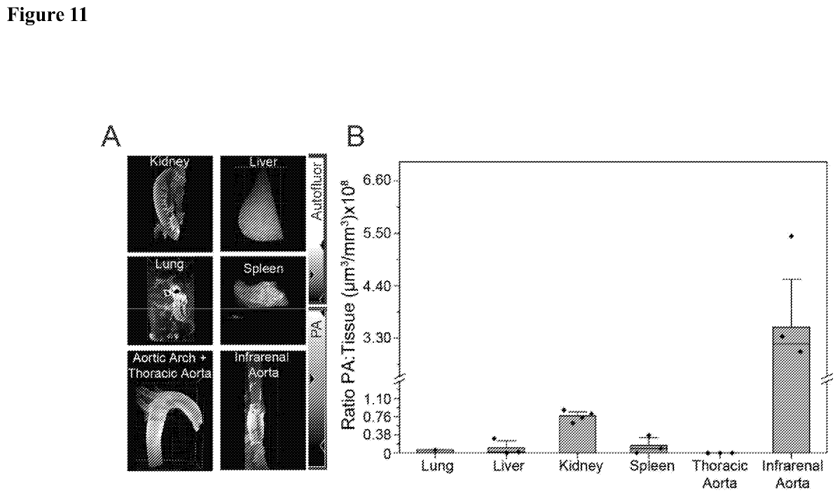

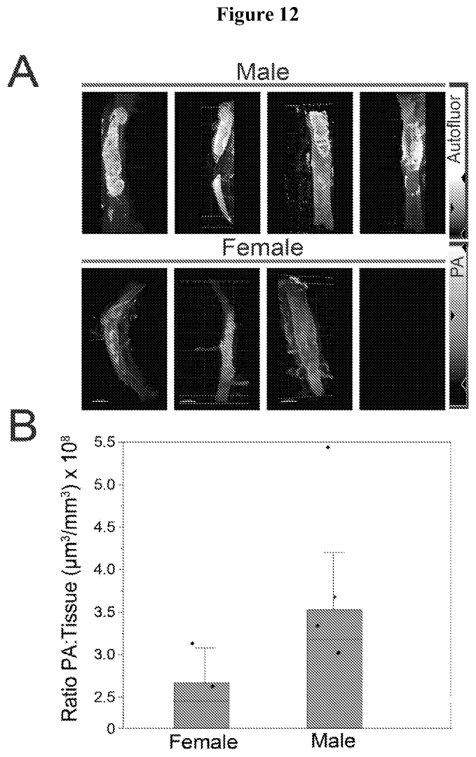

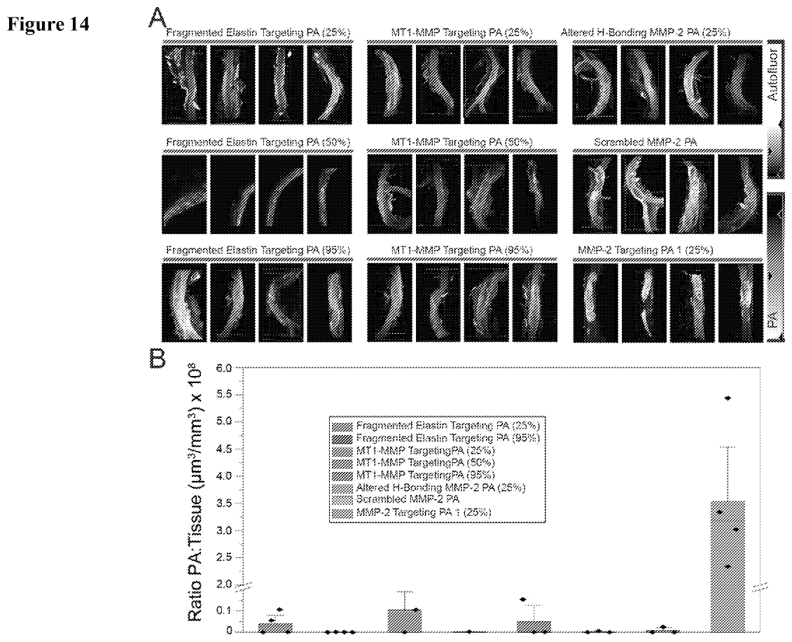

Results of the peptide amphiphile targeting study for fragmented elastin targeting PA, MT1-MMP targeting PA, MMP-2-targeting PA 1, and the scrambled MMP-2 PA control are shown in FIG. 8. FIG. 8A shows fragmented elastin targeting PA, MT1-MMP targeting PA, and MMP-2-targeting PA 1 targeting to the rat aorta. The ratio of PA volume to tissue volume for each AAA-targeting PA is shown in FIG. 8B. MMP-2-targeting PA 1 showed the highest ratio of PA volume to tissue volume. Additionally, MMP-2-targeting PA 1 predominantly localized to the aorta (FIG. 11). Moreover, differential localization of MMP-2 targeting PA is seen in male and female rats (FIG. 12). Complete LSFM of AAA targeting by various PAs is shown in FIG. 14.

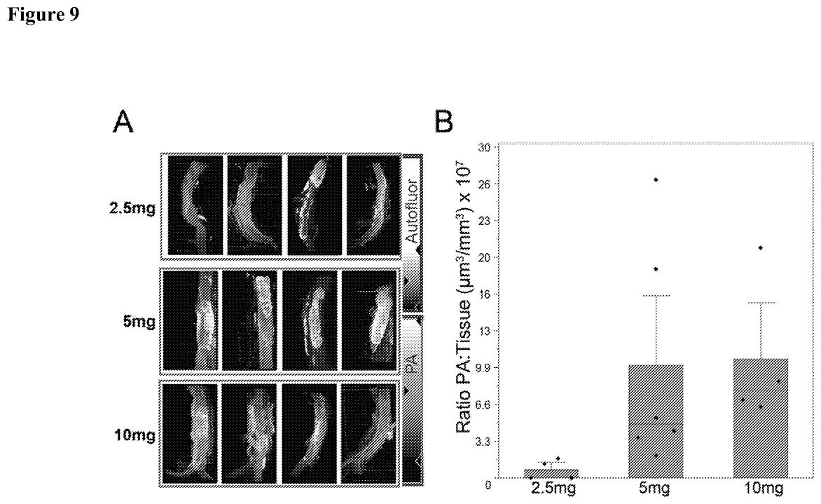

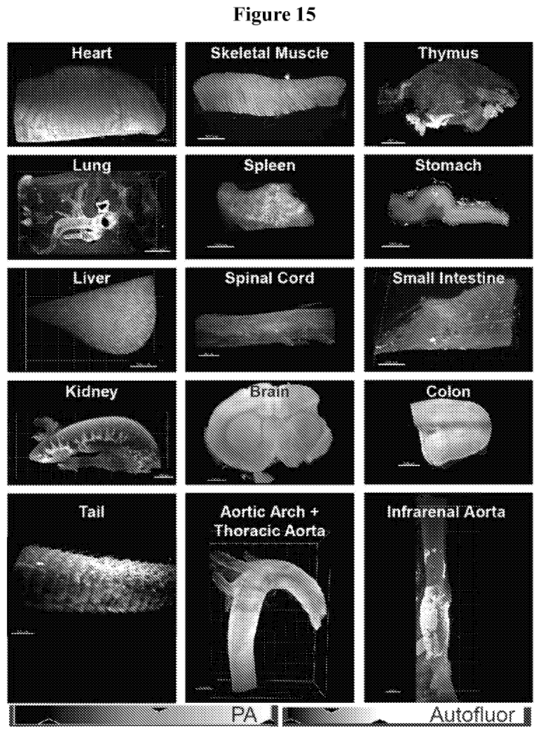

Targeting specificity of both MMP-2-targeting and elastin-targeting PAs to the aneurysmal aorta, non-aneurysmal suprarenal aorta, and all vital organs is assessed using in vivo imaging system (IVIS), light-sheet fluorescence microscopy (LSFM), and immunofluorescence (IF) microscopy. Preliminary data shows that the MMP-2-targeted PA nanomaterial localizes to sites of aneurysm formation in a dose dependent manner, but not to non-aneurysmal suprarenal aorta (FIG. 3). Representative LSFM images of all rat tissues examined for PA biodistribution are shown in FIG. 15. Targeting specificity was determined by measuring pixel count above an assigned threshold. Further, MMP-2 was quantified using IF microscopy and correlated to the amount of MMP-2-targeting PA. When no adverse side effects occurred, the dosage was increased until we achieved saturation of targeting. With this experiment, we confirmed that targeted PA nanomaterials localized to the site of interest and optimal dose for duration studies was determined. Targeting PA dosage optimization is shown in FIG. 9. Results are shown for 2 hours post PA injection of either 2.5 mg, 5 mg, or 10 mg of MMP-2-targeting PA 1. A higher PA volume to tissue volume ratio is seen in the 5 mg and 10 mg dose than the 2.5 mg dose. The 5 mg dose was selected for further investigation. Since the PA to tissue volume ratio was similar between the 5 mg and 10 mg dose, the binding is likely saturated at the 5 mg dose.

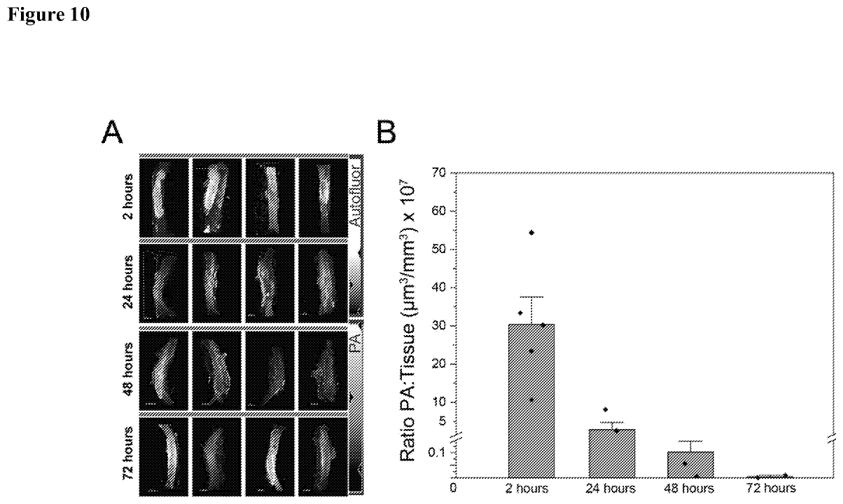

Duration of localization. Thirty-five days after AAA induction, rats will receive one of two treatments: 1) optimal dosage for MMP-2-targeting PA or 2) optimal dosage for elastin-targeting PA. The duration of localization is determined for the targeting PA nanomaterials in aneurysmal tissue by examining the aorta at, for example, 1, 2, 3, 4, and 5 days. MMP-2-targeting PA localization duration is shown in FIG. 10 for rats injected with 5 mg or 10 mg of MMP-2-targeting PA 1. An assessment of MMP-2-targeting PA localization was made at 2 hours, 24 hours, 48 hours, and 72 hours post PA injection. The highest ratio of PA volume to tissue volume is seen at 2 hours post PA injection.

Minimal targeting of fragmented elastin-targeting PAs or only transient localization of either targeting PA may be encountered. Potential reasons include improper N-terminal to C-terminal epitope orientation, not enough space between p sheet region of the PA and the epitope, or perhaps sub-optimal PA formulation. In embodiments, for epitope orientation issues, an elastin-targeting PA on C₁₆V₂A₂E₂(SEQ ID NO: 5) in place of an E₂A₂V₂K(C₁₂) (SEQ ID NO: 6) diluent PA is synthesized to alter the N-terminal to C-terminal epitope orientation of the PA. In embodiments, if any issues arise with 3D hindrance of the epitope, a longer glycine spacer or a polyethylene glycol (PEG) spacer is used between the β-sheet region and epitope.

### Example 4: Examine the Biocompatibility and Safety of the Targeted PA Nanomaterials In Vivo

After determining the optimal dose that results in the most specific targeting to aneurysmal tissue, the biocompatibility of the targeted PA nanomaterials is evaluated. Specifically, the aorta and all vital organs are examined for inflammation and architectural changes via IF and histology. Blood is assayed at multiple time points before and after administration of the targeted PA nanomaterial to assess cytokine release, complement activation, the coagulation cascade, and routine blood chemistries. Lastly, vital signs, weight, and overall animal behavior are closely monitored.

To avoid unnecessary animal usage in future efficacy studies, biocompatibility & safety is examined.

Biocompatibility. To evaluate PA nanomaterial biocompatibility, AAA induced rats are given the optimal concentration, dose, and co-assembly ratio of one of the following 3 treatments via intravenous injection (N=8 rats, 4 males and 4 females, per group): 1) MMP-2-targeting PA 1, 2) C₁₆-V₂A₂E₂ (SEQ ID NO: 5) diluent PA, and 3) saline. Animals are sacrificed at 2 hours, 1 day, 3 days, 1 and 4 weeks after injection. The aorta and all vital organs are examined for inflammation via IF staining (CD68 for monocytes and myeloperoxidase for neutrophils) and architectural changes via histology (hemoatoxylin and eosin staining). Rats are monitored daily for hunching, grimace, and lack of activity.

Systemic safety. To assess safety of PA nanomaterials, animals with AAA induced as described above are given the optimal concentration, dose, and co-assembly ratio of one of the following 3 treatments via intravenous injection (N=8 rats, 4 males and 4 females, per treatment group): 1) MMP-2-targeting PA 1, 2) C₁₆-V₂A₂E₂ (SEQ ID NO: 5) diluent PA, and 3) saline. Animals are sacrificed at 2 hours, 1 and 3 days after injection. Blood collected before and after PA administration is analyzed for hematologic (CBC, platelets, PT/PTT) and clinical chemistries (Chem-7, amylase, lipase), including liver tests for aspartate aminotransferase and alanine aminotransferase. Complement activation is assessed using an ELISA from Abcam according to manufacturer's instructions. Cytokine profile is assessed using an ELISA.

If toxicity is encountered, a solution is to use multiple drying steps on the Schlenk line to remove any potential adsorbed HFIP. In embodiments, the dosage is decreased until toxicity ceases, but still targets. Additionally, in embodiments an alternative solvent to HFIP such as DMSO or TFA is used to disaggregate and co-assemble the PA. In embodiments, PA diluent is altered, the elastin- and MMP-2-targeting peptides are altered, and/or the other structural formulations such as PA-based micelles or lowering the charge of the diluent PA are altered. In embodiments, we may employ an alternative target protein.

Data is evaluated using 1- or 2-factor ANOVA and a post-hoc Tukey significant difference test using Origin statistical software. A p-value <0.05 indicates significance between test conditions. The number of animals used was calculated using a power analysis with 20% minimal detection difference, an error rate less than 5%, and a 10% standard deviation estimated from previous Sprague Dawley rats.

### Example 5: Development of Intravenously Injectable Nanomaterial to Target Aortic Aneurysms

We develop an intravenously injectable nanomaterial to target aortic aneurysms, i.e., a nanoscale drug delivery system comprised of peptide amphiphile (PA) nanomaterials that can be injected intravenously and target the aneurysmal microenvironment. These nanomaterials are comprised of a synthetic peptide covalently attached to an aliphatic molecule. The peptide consists of a β-sheet forming unit, a charged unit, and an optional epitope region. PA molecules spontaneously form nanomaterials in an aqueous environment. Using specific epitopes, we can target proteins of interest. The nanofiber structure increases the surface area available to interact with target proteins which in many cases improves avidity of localization to targeted proteins. Additionally, we can covalently incorporate therapeutics directly onto PA monomers and/or incorporate hydrophobic drugs into the aliphatic core of the nanofiber structure.

Use as a Delivery Vehicle: One particularly useful feature of PA nanomaterials is that they can be used both to target and to deliver therapeutics. Using multiple PA monomers, we can co-assemble with both targeting PAs and PA monomers containing covalently attached therapeutics, fluorescent tags, and/or radiocontrast agents.

Our PA molecules were designed with epitopes to target proteins overexpressed in aneurysms (matrix metalloproteinase 2 [MMP-2] and membrane type metalloproteinase 1 [MT1-MMP]), and fragmented elastin, which is a feature exclusive to pathological arteries.

In other embodiments of our PA nanomaterials, we have covalently incorporated nitric oxide-releasing molecules directly into the PA monomers, which were co-assembled with targeting monomers. Upon co-assembly these supramolecular nanomaterials targeted diseased tissue and activated localized release of nitric oxide. In embodiments, microbubbles, nanobubles, positron-emitting contrast agents and/or gadolinium contrast agents are incorporated into the PA structure. The contrast can be used to visualize very early stage aneurysms before significant dilation occurs. In embodiments, the PA nanomaterials disclosed herein is used in a method of detecting aneurysms. In embodiments, the aneurysm is an early stage aneurysm. In embodiments, the aneurysm is an early stage abdominal aortic aneurysm.

These molecules were synthesized using solid phase 9-fluorenylmethoxycarbonyl chemistry. They were then characterized with transmission electron microscopy (TEM) to confirm nanofiber structure. Using a well-established model of aortic aneurysm (CaCl₂-soaked sponge) we induced aneurysms in rats and tested targeting capabilities of our PA nanomaterials in Sprague Dawley rats. Three of the four PA molecules that we designed had high synthesis efficiency and produced nanomaterials. Using light sheet fluorescent microscopy and conventional fluorescent microscopy we observed localization of the MMP-2_RGA PA to aneurysmal tissue.

This platform can be used both as a targeted therapeutic using nitric oxide-releasing compounds and/or for very early detection of AAA.

Advantages over existing technologies: Currently there are no FDA-approved therapeutics that can significantly inhibit the dilation of aortic aneurysms. Employing PA nanomaterials to activate localized release of NO or to inhibit MMP degradation of medial elastin would constitute a revolutionary therapeutic for AAA. Additionally, current methods to detect aneurysms rely on ultrasound imaging, which only detects AAA after the artery has already undergone significant dilation. This PA nanomaterial platform can be used to detect high levels of MT1-MMP and MMP-2 before dilation has occurred. Thus, this platform can be used as a targeted therapeutic using nitric oxide-releasing compounds and/or for very early detection of AAA.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### BIBLIOGRAPHY

1. Davis F M, Rateri D L, Daugherty A. Abdominal aortic aneurysm: novel mechanisms and therapies. Curr Opin Cardiol. 2015; 30(6):566-73. doi: 10.1097/HCO.0000000000000216. PubMed PMID: 26352243; PMCID: PMC4624089.
2. Didangelos A, Yin X, Mandal K, Saje A, Smith A, Xu Q, Jahangiri M, Mayr M. Extracellular matrix composition and remodeling in human abdominal aortic aneurysms: a proteomics approach. Mol Cell Proteomics. 2011; 10(8):M111 008128. doi: 10.1074/mcp.M111.008128. PubMed PMID: 21593211; PMCID: PMC3149094.
3. Freestone T, Turner R J, Coady A, Higman D J, Greenhalgh R M, Powell J T. Inflammation and matrix metalloproteinases in the enlarging abdominal aortic aneurysm. Arterioscler Thromb Vasc Biol. 1995; 15(8):1145-51. PubMed PMID: 7627708.
4. Longo G M, Xiong W, Greiner T C, Zhao Y, Fiotti N, Baxter B T. Matrix metalloproteinases 2 and 9 work in concert to produce aortic aneurysms. J Clin Invest. 2002; 110(5):625-32. Epub 2002/09/05. doi: 10.1172/JCI15334. PubMed PMID: 12208863; PMCID: PMC151106.
5. Pharmaceuticals NPN. ACZ885 for the Treatment of Abdominal Aortic Aneurysm (AAA) Clinicaltrials.gov2016 [updated Nov. 21, 2016; cited 2018 Oct. 31]. Available from: https://clinicaltrials.gov/ct2/show/study/NCT02007252?rslt=With&type=Intr&cond=Aortic+Aneurysm&rank=1&sect=X370156.
6. Keisler B, Carter C. Abdominal aortic aneurysm. Am Fam Physician. 2015; 91(8):538-43. PubMed PMID: 25884861.
7. Henderson E L, Geng Y J, Sukhova G K, Whittemore A D, Knox J, Libby P. Death of smooth muscle cells and expression of mediators of apoptosis by T lymphocytes in human abdominal aortic aneurysms. Circulation. 1999; 99(1):96-104. Epub 1999/01/13. PubMed PMID: 9884385.
8. Lopez-Candales A, Holmes D R, Liao S, Scott M J, Wickline S A, Thompson R W. Decreased vascular smooth muscle cell density in medial degeneration of human abdominal aortic aneurysms. Am J Pathol. 1997; 150(3):993-1007. Epub 1997/03/01. PubMed PMID: 9060837; PMCID: PMC1857880.
9. Chan C Y T, Cheuk B L Y, Cheng S W K. Abdominal Aortic Aneurysm-Associated MicroRNA-516a-5p Regulates Expressions of Methylenetetrahydrofolate Reductase, Matrix Metalloproteinase-2, and Tissue Inhibitor of Matrix Metalloproteinase-1 in Human Abdominal Aortic Vascular Smooth Muscle Cells. Ann Vasc Surg. 2017; 42:263-73. doi: 10.1016/j.avsg.2016.10.062. PubMed PMID: 28288890.
10. Ciavarella C, Alviano F, Gallitto E, Ricci F, Buzzi M, Velati C, Stella A, Freyrie A, Pasquinelli G. Human Vascular Wall Mesenchymal Stromal Cells Contribute to Abdominal Aortic Aneurysm Pathogenesis Through an Impaired Immunomodulatory Activity and Increased Levels of Matrix Metalloproteinase-9. Circ J. 2015; 79(7):1460-9. doi: 10.1253/circj.CJ-14-0857. PubMed PMID: 25854712.
11. CDC. Aortic Aneurysm Fact Sheet 2016 [updated Jun. 16, 2016; cited 2018 10/30]. Available from: https://www.cdc.gov/dhdsp/data_statistics/fact_sheets/fs_aortic_aneurysm.htm.
12. Annambhotla S, Bourgeois S, Wang X, Lin P H, Yao Q, Chen C. Recent advances in molecular mechanisms of abdominal aortic aneurysm formation. World J Surg. 2008; 32(6):976-86. doi: 10.1007/s00268-007-9456-x. PubMed PMID: 18259804; PMCID: PMC2927355.
13. Kimura T, Yoshimura K, Aoki H, Imanaka-Yoshida K, Yoshida T, Ikeda Y, Morikage N, Endo H, Hamano K, Imaizumi T, Hiroe M, Aonuma K, Matsuzaki M. Tenascin-C is expressed in abdominal aortic aneurysm tissue with an active degradation process. Pathol Int. 2011; 61(10):559-64. doi: 10.1111/j.1440-1827.2011.02699.x. PubMed PMID: 21951663.
14. Stather P W, Sidloff D A, Dattani N, Gokani V J, Choke E, Sayers R D, Bown M J. Metaanalysis and meta-regression analysis of biomarkers for abdominal aortic aneurysm. Br J Surg. 2014; 101(11):1358-72. doi: 10.1002/bjs.9593. PubMed PMID: 25131707.
15. Vasic N, Glumac S, Pejic S, Amidzic L J, Tadic Latinovic L J, Dozic B, Hinic S, Maksimovic Z. Expression of Matrix Metalloproteinases and Endogenous Inhibitors in Abdominal Aortic Aneurysm and Aortoiliac Occlusive Disease (Syndrome Leriche). Folia Biol (Praha). 2017; 63(5-6):209-16. PubMed PMID: 29687775.
16. Moran C S, McCann M, Karan M, Norman P, Ketheesan N, Golledge J. Association of osteoprotegerin with human abdominal aortic aneurysm progression. Circulation. 2005; 111(23):3119-25. doi: 10.1161/CIRCULATIONAHA.104.464727. PubMed PMID: 15939823.
17. Okada Y, Katsuda S, Okada Y, Nakanishi I. An elastinolytic enzyme detected in the culture medium of human arterial smooth muscle cells. Cell Biol Int. 1993; 17(9):863-9. Epub 1993/09/01. doi: 10.1006/cbir.1993.1149. PubMed PMID: 8220313.
18. Gertz S D, Kurgan A, Eisenberg D. Aneurysm of the rabbit common carotid artery induced by periarterial application of calcium chloride in vivo. J Clin Invest. 1988; 81(3):649-56. doi: 10.1172/JCI113368. PubMed PMID: 3343336; PMCID: PMC442510.
19. Klaus V, Tanios-Schmies F, Reeps C, Trenner M, Matevossian E, Eckstein H H, Pelisek J. Association of Matrix Metalloproteinase Levels with Collagen Degradation in the Context of Abdominal Aortic Aneurysm. Eur J Vasc Endovasc Surg. 2017; 53(4):549-58. doi: 10.1016/j.ejvs.2016.12.030. PubMed PMID: 28209269.
20. Siennicka A, Zuchowski M, Kaczmarczyk M, Cnotliwy M, Clark J S, Jastrzebska M. Spatial differences of matrix metalloproteinase-2 and matrix metalloproteinase-9 within abdominal aortic aneurysm wall and intraluminal thrombus. J Physiol Pharmacol. 2016; 67(6):903-10. PubMed PMID: 28195071.
21. Davis V, Persidskaia R, Baca-Regen L, Itoh Y, Nagase H, Persidsky Y, Ghorpade A, Baxter BT. Matrix metalloproteinase-2 production and its binding to the matrix are increased in abdominal aortic aneurysms. Arterioscler Thromb Vasc Biol. 1998; 18(10):1625-33. Epub 1998/10/09. PubMed PMID: 9763536.
22. Morgan C E, Dombrowski A W, Rubert Perez C M, Bahnson ES , Tsihlis N D, Jiang W, Jiang Q, Vercammen J M, Prakash V S, Pritts T A, Stupp S I, Kibbe M R. Tissue-Factor Targeted Peptide Amphiphile Nanofibers as an Injectable Therapy To Control Hemorrhage. ACS Nano. 2016; 10(1):899-909. doi: 10.1021/acsnano.5b06025. PubMed PMID: 26700464.
23. Moyer T J, Kassam H A, Bahnson E S, Morgan C E, Tantakitti F, Chew T L, Kibbe M R, Stupp S I. Shape-Dependent Targeting of Injured Blood Vessels by Peptide Amphiphile Supramolecular Nanostructures. Small. 2015; 11(23):2750-5. doi: 10.1002/smll.201403429. PubMed PMID: 25649528; PMCID: PMC4478239.
24. Bahnson E S, Kassam H A, Moyer T J, Jiang W, Morgan C E, Vercammen J M, Jiang Q, Flynn M E, Stupp S I, Kibbe M R. Targeted Nitric Oxide Delivery by Supramolecular Nanofibers for the Prevention of Restenosis After Arterial Injury. Antioxid Redox Signal. 2016; 24(8):401-18. Epub 2015/11/26. doi: 10.1089/ars.2015.6363. PubMed PMID: 26593400; PMCID: PMC4782035.
25. Aggarwal S, Qamar A, Sharma V, Sharma A. Abdominal aortic aneurysm: A comprehensive review. Exp Clin Cardiol. 2011; 16(1):11-5. PubMed PMID: 21523201; PMCID: PMC3076160.
26. Sinha A, Shaporev A, Nosoudi N, Lei Y, Vertegel A, Lessner S, Vyavahare N. Nanoparticle targeting to diseased vasculature for imaging and therapy. Nanomedicine. 2014; 10(5):1003-12. doi: 10.1016/j.nano.2014.02.002. PubMed PMID: 24566276; PMCID: PMC4077993.
27. Blanchevoye C, Floquet N, Scandolera A, Baud S, Maurice P, Bocquet O, Blaise S, Ghoneim C, Cantarelli B, Delacoux F, Dauchez M, Efremov R G, Martiny L, Duca L, Debelle L. Interaction between the elastin peptide VGVAPG and human elastin binding protein. J Biol Chem. 2013; 288(2):1317-28. doi: 10.1074/jbc.M112.419929. PubMed PMID: 23166321; PMCID: PMC3543015.
28. Hinek A, Rabinovitch M. 67-kD elastin-binding protein is a protective “companion” of extracellular insoluble elastin and intracellular tropoelastin. J Cell Biol. 1994; 126(2):563-74. PubMed PMID: 8034752; PMCID: PMC2200028.
29. Hinek A, Smith A C, Cutiongco E M, Callahan J W, Gripp K W, Weksberg R. Decreased elastin deposition and high proliferation of fibroblasts from Costello syndrome are related to functional deficiency in the 67-kD elastin-binding protein. Am J Hum Genet. 2000; 66(3):859-72. doi: 10.1086/302829. PubMed PMID: 10712202; PMCID: PMC1288169.
30. Hinek A, Mecham R P, Keeley F, Rabinovitch M. Impaired elastin fiber assembly related to reduced 67-kD elastin-binding protein in fetal lamb ductus arteriosus and in cultured aortic smooth muscle cells treated with chondroitin sulfate. J Clin Invest. 1991; 88(6):2083-94. doi: 10.1172/JCI115538. PubMed PMID: 1661296; PMCID: PMC295807.
31. Hinek A, Pshezhetsky A V, von Itzstein M, Starcher B. Lysosomal sialidase (neuraminidase-1) is targeted to the cell surface in a multiprotein complex that facilitates elastic fiber assembly. J Biol Chem. 2006; 281(6):3698-710. doi: 10.1074/jbc.M508736200. PubMed PMID: 16314420.
32. Morgunova E, Tuuttila A, Bergmann U, Tryggvason K. Structural insight into the complex formation of latent matrix metalloproteinase 2 with tissue inhibitor of metalloproteinase 2. Proc Natl Acad Sci USA. 2002; 99(11):7414-9. doi: 10.1073/pnas.102185399. PubMed PMID: 12032297; PMCID: PMC124245.
33. Sato K, Ji W, Palmer L C, Weber B, Barz M, Stupp S I. Programmable Assembly of Peptide Amphiphile via Noncovalent-to-Covalent Bond Conversion. J Am Chem Soc. 2017; 139(26):8995-9000. doi: 10.1021/jacs.7b03878. PubMed PMID: 28639790; PMCID: PMC5553714.
34. Hubmacher D, Bergeron E, Fagotto-Kaufmann C, Sakai L Y, Reinhardt D P. Early fibrillin-1 assembly monitored through a modifiable recombinant cell approach. Biomacromolecules. 2014; 15(4):1456-68. Epub 2014/02/25. doi: 10.1021/bm5000696. PubMed PMID: 24559401; PMCID: PMC4961472.
35. Koole D, Hurks R, Schoneveld A, Vink A, Golledge J, Moran C S, de Kleijn D P, van Herwaarden J A, de Vries J P, Laman J D, Huizinga R, Pasterkamp G, Moll F L. Osteoprotegerin is associated with aneurysm diameter and proteolysis in abdominal aortic aneurysm disease. Arterioscler Thromb Vasc Biol. 2012; 32(6):1497-504. doi: 10.1161/ATVBAHA.111.243592. PubMed PMID: 22516062.

1. A peptide amphiphile comprising: (a) a hydrophobic non-peptidic segment; (b) a β-sheet-forming peptide segment; (c) a charged peptide segment; (d) a targeting moiety, wherein the targeting moiety localizes to MMP-2, MT1-MMP, or fragmented elastin; and optionally (e) a therapeutic agent; wherein the hydrophobic non-peptidic segment is covalently attached to the N-terminus of the β-sheet-forming peptide segment; wherein the 0-sheet-forming peptide segment is covalently attached to the targeting moiety; and wherein the charged peptide segment is covalently attached to the targeting moiety.

2. The peptide amphiphile of claim 1, wherein said targeting moiety comprises a peptide capable of targeting an epitope of MMP-2, an epitope of MT1-MMP, or an epitope of fragmented elastin.

3. The peptide amphiphile of claim 2, wherein said targeting moiety is a peptide that is capable of targeting an epitope on MMP-2.

4. The peptide amphiphile of claim 3, wherein said peptide comprises a sequence with at least 80% homology to SEQ ID NO: 1.

5. The peptide amphiphile of claim 2, wherein said targeting moiety is a peptide that is capable of targeting an epitope on MT1-MMP.

6. The peptide amphiphile of claim 5, wherein said peptide comprises a sequence with at least 80% homology to SEQ ID NO: 3.

7. The peptide amphiphile of claim 2, wherein said targeting moiety comprises a peptide that is capable of targeting an epitope on fragmented elastin.

8. The peptide amphiphile of claim 1, further comprising a therapeutic agent.

9. The peptide amphiphile of claim 8, wherein the therapeutic agent is selected from the group consisting of nitric oxide, an angiotensin receptor blocker, an ACE inhibitor, an MMP inhibitor, and a TGF-β agonist.

10-13. (canceled)

14. The peptide amphiphile of claim 1, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

15. A self-assembled nanomaterial comprising:
a plurality of peptide amphiphiles of claim 1.

16. The self-assembled nanomaterial of claim 15 wherein said targeting moiety comprises a peptide capable of targeting an epitope of MMP-2, an epitope of MT1-MMP, or an epitope of fragmented elastin.

17-21. (canceled)

22. The self-assembled nanomaterial of claim 15,
further comprising a therapeutic agent.

23. The self-assembled nanomaterial of claim 22, wherein the therapeutic agent is selected from the group consisting of nitric oxide, an angiotensin receptor blocker, an ACE inhibitor, an MMP inhibitor, and a TGF-β agonist.

24-27. (canceled)

28. The self-assembled nanomaterial of claim 15, wherein the C-terminus of the β-sheet-forming peptide segment is covalently attached to the N-terminus of the charged peptide segment; and wherein the C-terminus of the charged peptide segment is covalently attached to the N-terminus of the targeting moiety.

29. A method of inhibiting the dilation of an aneurysm in a subject comprising, administering to the subject a composition comprising a self-assembled nanomaterial comprising:
a plurality of peptide amphiphiles, wherein said peptide amphiphiles comprise:
(a) a hydrophobic non-peptidic segment;
(b) a β-sheet-forming peptide segment;
(c) a charged peptide segment; and
(d) a targeting moiety,
wherein the targeting moiety targets MMP-2 or MT1-MMP or fragmented elastin;
wherein the hydrophobic non-peptidic segment is covalently attached to the N-terminus of the β-sheet-forming peptide segment; wherein the β-sheet-forming peptide segment is covalently attached to the targeting moiety; and wherein the charged peptide segment is covalently attached to the targeting moiety.

30-32. (canceled)

33. The method of claim 29, wherein the peptide amphiphile further comprises a therapeutic agent.

34. A method of delivering a therapeutic agent to an aneurysm site in a subject comprising, administering to the subject a composition comprising the self-assembled nanomaterial of claim 15.

35. A method of making a peptide amphiphile (PA)-based nanomaterial which targets MMP-2, MT1-MMP, or fragmented elastin comprising:
synthesizing targeting PA molecules via solid phase peptide synthesis comprising contacting an elastin-targeting peptide, MMP-2-targeting peptide, or MT1-MMP-targeting peptide with a diluent PA backbone;
purifying the PA molecules;
dissolving targeting PA molecules and with a diluent PA in a molar ratio in a solvent;
removing the solvent; and
forming the nanomaterial via self-assembly by resuspending the mixture of PA molecules in liquid at physiological pH.

36-42. (canceled)

43. The method of claim 35, wherein the elastin-targeting peptide, MMP-2-targeting peptide, or MT1-MMP-targeting peptide is connected to the diluent PA backbone by a covalent bond in the resulting targeting PA molecule.
